(19) United States
(12) Patent Application Publication
(10) Pub. No.: US 2024/0410902 A1
(43) Pub. Date: Dec. 12, 2024

(54) USE OF SOLUBLE TREM2 AS A NON-INVASIVE BIOMARKER FOR NON-ALCOHOLIC STEATOHEPATITIS (NASH)

(71) Applicants: Syddansk Universitet, Odense M (DK); Region Syddanmark, Vejle (DK)

(72) Inventors: Niels Jonas Heilskov GRAVERSEN, Odense M (DK); Vineesh Indira CHANDRAN, Odense M (DK); Søren Kragh MOESTRUP, Aarhus N (DK); Maria Kløjgaard SKYTTHE, Odense C (DK); Aleksander Ahm KRAG, Odense M (DK); Charlotte WERNBERG, Esbjerg (DK); Mette Enok Munk LAURIDSEN, Odense M (DK)

(21) Appl. No.: 18/564,767

(22) PCT Filed: May 20, 2022
(86) PCT No.: PCT/EP2022/063769

§ 371 Date: Nov. 28, 2023

(30) Foreign Application Priority Data
May 28, 2021 (EP) 21176575.5

(51) Int. Cl. G01N 33/68; A61K 45/06

(52) U.S. Cl. CPC G01N 33/6893; A61K 45/06; G01N 2333/70503; G01N 2800/085; G01N 2800/50; G01N 2800/52

(57) ABSTRACT

Reliable non-invasive biomarkers are an unmet clinical need for the diagnosis of NASH patients. Herein we identify soluble TREM2 as a non-invasive plasma biomarker to diagnose NASH in obese patients and provides evidence for sTREM2 as an accurate predictor of advanced hepatic inflammation, ballooning, and fibrosis.

### TECHNICAL FIELD OF THE INVENTION

The present invention relates to the identification of Triggering receptor expressed on myeloid cells 2 (TREM2) being a biomarker for non-alcoholic steatohepatitis (NASH). In particular, the present invention relates to TREM2 found in blood, plasma or serum, known as soluble TREM2 (sTREM2), as a biomarker for NASH.

### BACKGROUND OF THE INVENTION

Non-alcoholic fatty liver disease (NAFLD) affects approximately 24% of adult population worldwide. The progression of NAFLD to the more severe disease, NASH, is accompanied by hepatocyte ballooning, hepatic necroinflammation, and fibrosis advancing to end-stage liver diseases (1). Currently, liver biopsy is the mainstay tool to diagnose NASH (2). However, the utility of liver biopsy in diagnosis is severely limited by its inherent invasive nature, biased sample representation and risk of bleeding complications leading to poor patient acceptability (3). Meanwhile, a plethora of non-invasive blood biomarkers have been assessed for their diagnostic accuracy in NASH, but none of these have qualified for routine clinical use partly due to the overall modest accuracy in independent validation (3). Thus, there is an urgent unmet clinical need for new non-invasive, point-of-care biomarkers for diagnosis and selection of patients for treatment and monitoring in NASH.

Liver macrophages, Kupffer cells and monocyte-derived macrophages, play a central role in the etiology of hepatic inflammation in NASH. There is widespread recognition that ability to detect hepatic inflammation may facilitate rapid and accurate diagnosis of NASH. TREMs is a protein class of cell surface receptors of the Ig superfamily that have been shown to mediate multiple pathophysiological processes in various diseases. Importantly, a multitude of recent studies have demonstrated the participation of TREM2 in inflammation-associated pathologies in the liver. For instance, TREM2 expression is upregulated in non-parenchymal hepatic cells including macrophages in both mouse and human samples during liver injury. Increased TREM2 expression in hepatic macrophages was also found strongly associated with higher NAFLD activity scores (NAS) reflecting its relevance in steatosis, inflammation, hepatocyte ballooning, and fibrosis (4). In another set of three independent studies, the authors identified a distinct TREM2-positive macrophage subpopulation that was associated with advanced stages of NASH (5-7). Recently, an integrated analysis of cirrhotic and healthy liver samples single cell RNA-seq data showed enrichment of TREM2 during NAFLD progression, particularly associated with advanced NASH (8). Taken together, there is strong evidence correlating increased expression of TREM2 on liver macrophages with severity of NASH pathogenesis.

The above-described expression of TREM2 was measured by invasive methods like liver biopsies from the subject. Further, it was TREM2 expressed on the macrophages that was quantified.

However, TREM2 regulation is a complicated mechanism including several layers of regulation.

A review by Zhong et al (Front. Aging Neurosci., 26 Nov. 2019) describes that “very little is known with regards to the mechanisms underlying sTREM2 generation under physiological and pathological conditions” in relation to Alzheimers disease.

Deming et al. (Sci Transl Med. 2019 Aug. 14; 11 (505) shows that increased levels of TREM2 is linked to increased expression of the MS4A gene cluster. Thus, suggesting that MS4A4A modulates sTREM2.

Feuerbach et al. (Neurosci Lett. 2017 Nov. 1; 660:109-114) states that an interplay between the extracellular portions TREM2 and DAP12 that could regulate activation and shedding of TREM2.

Berner et al. The FASEB Journal. 2020; 34:6675-6687 finds that meprin β sheds and degrades TREM2 at the cell surface.

Hence, a more accurate non-invasive method for identifying individuals at risk for developing or having NASH is an urgent clinical need of high significance, and in particular a more efficient and/or reliable non-invasive method for following the effect of treatment in NASH patients would be advantageous.

### SUMMARY OF THE INVENTION

In here, it has been realized that sTREM2 can be exploited as a non-invasive biomarker of cellular TREM2 expression in detecting patients with NASH. Therefore, motivated by clinical need, in here is presented biochemical ELISA and tissue analysis in a histologically well-defined discovery cohort of patients to explore the clinical utility of TREM2 as a non-invasive biomarker to distinguish NASH patients from obese controls.

Example 1 shows that TREM2 is highly expressed on macrophages in the liver of patients diagnosed with NASH compared to healthy controls, measured as mRNA levels of Trem2 (FIG. 2). However, TREM2 expression or density on macrophages can only be measured using invasive methods such as liver biopsies.

Example 2 shows that sTREM2 levels in blood can distinguish NASH patients from obese controls including subjects with NAFLD (FIG. 3).

Thus, an object of the present invention relates to the use of sTREM2 levels in blood as a non-invasive biomarker in diagnosing NASH.

In particular, it is an object of the present invention to provide a method based on the sTREM2 levels in the blood that can be used to either follow the outcome of a treatment in NASH patients or to follow the progression of the disease.

Thus, one aspect of the invention relates to a method for determining the risk of a subject having or developing NASH, the method comprising
A) determining in a blood sample from a subject, the level of soluble TREM2 (sTREM2);
B) comparing said determined level to a reference level; and
   determining that said subject is at risk of having or developing NASH, if said level is above said reference level, or
   determining that said subject is not at risk of having or developing NASH if said level is equal to or below said reference level.

Preferably, the method is for determining the risk of a subject having NASH.

Another aspect of the present invention relates to a method for monitoring the development of NASH in a subject, the method comprising
determining a first level of sTREM2 in a first blood sample from the subject;
determining a second level of sTREM2 in a second blood sample from the subject, wherein the second sample has been obtained at a later time point than the first sample;
comparing corresponding levels in the first and second sample;
wherein
   a higher sTREM2 level in the second sample compared to the first sample is indicative of a worse NASH;
   a equal or lower sTREM2 level in the second sample compared to the first sample is indicative of unchanged or improvement in NASH.

Yet another aspect of the present invention a method for determining the effect of a treatment protocol against NASH for a subject, the method comprising
determining a first level of sTREM2 in a first blood sample from the subject;
determining a second level of sTREM2 in a second blood sample from the subject, wherein the second sample has been obtained at a later time point than the first sample;
wherein the treatment protocol has been initiated or completed before the sampling of the first sample or initiated, continued or completed between the sampling of the first and second sample,
comparing sTREM2 levels in the first sample and the second sample; wherein
   a sTREM2 level in the second sample below or equal to the sTREM2 level in the first sample is indicative of the treatment protocol being efficient against NASH or,
   a sTREM2 level in the second sample above the sTREM2 level in the first sample is indicative of the treatment protocol not being efficient against NASH.

Still another aspect of the present invention relates to the use of blood sample levels of sTREM2 from a subject as a biomarker for the risk for said subject having or developing NASH or staging of NASH for said subject, preferably having NASH.

### BRIEF DESCRIPTION OF THE FIGURES

FIG. 1 shows (A) Graphical representation of TREM2 protein domains and its ectodomain cleavage site. SP, signal peptide; Ig, Immunoglobulin domain; Stalk domain; Tm, transmembrane domain; and Cyto, cytoplasmic domain. (B) Full length TREM2 undergoes regulated membrane proteolysis that is triggered via shedding of its ectodomain by disintegrin and metalloproteases such as ADAM10 and ADAM17 at His157-Ser158 to generate a soluble form of TREM2 that can be found in plasma.

FIG. 2 shows (A) Trem2 count in NASH patients and no-NASH controls obtained by analysing publicly available RNA seq data from 216 liver biopsies and categorized according to NAFLD activity score (NAS) (≥3, NASH and <3, no-NASH) (8). (B) Diagnostic accuracy of Trem2 count between NASH patients and no-NASH controls according to NAS.

FIG. 3 shows that soluble TREM2 (sTREM2) levels can discriminate NASH from no-NASH patients. (A), Quantitative ELISA analysis of TREM2 levels in plasma extracted from NASH patients and Obese controls. (B) Cumulative representation of quantitative ELISA data from (A). (C) sTREM2 levels in NASH patients and no-NASH controls with dichotomization according to NAFLD activity score (NAS) (≥3, NASH and <3, no-NASH). (D) Diagnostic accuracy of sTREM2 between NASH patients and no-NASH controls according to NAS. (E) sTREM2 levels in NASH patients and no-NASH controls with dichotomization according to SAF activity score (SAS) (>2, NASH and ≤2, no-NASH). (F) Diagnostic accuracy of sTREM2 between NASH patients and no-NASH controls according to SAS.

FIG. 4 shows that plasma sTREM2 levels are associated with NAFLD progression. Distribution of NASH patients and no-NASH controls according to NASH Clinical Research Network (NASH CRN) histologic scoring system for (A) steatosis, (B) ballooning, and (C) lobular inflammation. (D) Representative H&E staining images show severity of ballooning (grey color arrow) and inflammation (black color arrow) associated histology in matched patient liver tissues and their corresponding sTREM2 concentrations. Scale bar, 20 μm.

FIG. 5 shows plasma sTREM2 levels can accurately diagnose advanced fibrosis patients. (A) Dichotomization of NASH patients and no-NASH controls according to Kleiner fibrosis grades (F0 (no fibrosis), F1 (portal), F2 (periportal), F3 (bridging fibrosis), and F4 (cirrhosis)). Diagnostic accuracy of sTREM2 between minimal and advanced fibrosis patients. (B) Dichotomization of NASH patients and no-NASH controls according to FibroScan fibrosis scores (no to mild fibrosis (2-8 kPa), moderate fibrosis (8-12 kPa), and advanced fibrosis (>12 kPa)). Diagnostic accuracy of sTREM2 between no to mild and advanced fibrosis patients. (C) Dichotomization of NASH patients and no-NASH controls according to aspartate transaminase to platelet ratio index (APRI) scores (no-fibrosis (<0.5), mild fibrosis (0.5-0.98), and advanced fibrosis (>0.98)). Diagnostic accuracy of sTREM2 between mild to advanced and no fibrosis patients. (D) Dichotomization of NASH patients and no-NASH controls according to fibrosis-4 index (FIB-4) scores (mild fibrosis (<1.45), moderate fibrosis (1.45-3.25), and advanced fibrosis (>3.25)). Diagnostic accuracy of sTREM2 between moderate and advanced fibrosis patients. (E) Representative Sirius red staining images of matched patient liver tissues showing no-, moderate-, and advanced fibrosis-related histopathology (red color) and their corresponding sTREM2 concentrations. Scale bar, 5 μm.

FIG. 6 shows sTREM2 level does not discriminate between (A) Age (median 50 years), (B) Gender, and (C) Body mass index (median 42.6).

FIG. 7 shows quantitative ELISA analysis of (A) TIMP1, (B) TIMP2, and (C) MMP2 levels in plasma extracted from NASH patients and Obese controls. (D) Table associated with Receiver Operating Characteristic (ROC) curve analysis depicted in A, B & C.

FIG. 8 shows correlation curves showing correlation of sTREM2 with steatosis, Ballooning and Inflammation in NASH patients as compared with no-NASH patients. (A) Steatosis, (B) Ballooning and (C) Inflammation in NASH patients as compared with no-NASH patients. (D) ROC curve showing diagnostic accuracy of sTREM2 between NAFLD patients with insulin resistance and NALFD patients without insulin resistance.

FIG. 9 shows (A) serum sTREM2 levels in NASH patients as compared with no-NASH control groups. (B) ROC curve showing diagnostic accuracy of sTREM2.

FIG. 10 shows how SAF score categorization can be used to distinguish between No-NAFLD, NAFLD and NASH.

FIG. 11 shows Plasma TREM2 levels can identify NASH patients from NAFLD activity score, NAS<4 subjects in a large validation study (n=170). (A) Quantitative ELISA analysis of TREM2 levels in plasma extracted from NAS>4 patients and NAS<4 subjects categorized according to NAS. (B) Diagnostic accuracy of plasma TREM2 levels in NAS>4 and NAS<4 patients.

FIG. 12 shows plasma TREM2 levels dual cut-off for NASH diagnosis. Plasma TREM2 levels according to NAFLD activity score (0-8) with dual cut-off rule-in NAS≥4 (≥65 ng/ml) and rule-out NAS<4 (≤38 ng/ml) patients and diagnostic accuracy test probabilities for plasma TREM2 with 95% CIs for NAFLD Activity score, NAS≥4 (at-risk NASH), with 2×2 tables.

FIG. 13 shows diagnostic accuracy of plasma TREM2 between at-risk NASH patients and no-risk patients according to the following classification: (A), At-risk NASH defined as: NAS≥4 +F≥2 and all other combinations as no-risk (NAS<4+F≥2, and NAS≥4+F<2, and NAS<4+F<2); (B). Strict NASH, according to SAF-definition (FLIP algorithm), defined as: Steatosis≥1, Inflammation≥1, Ballooning≥1 and all other combinations as simple NASH (steatosis<1 (no NAFLD with either inflammation or ballooning or none) and steatosis≥1 (NAFLD with either inflammation or ballooning or none)).

FIG. 14 shows diagnostic accuracy of plasma TREM2 between at-risk NASH patients and no-risk patients according to the following classification compared to composite biomarkers of NASH.

FIG. 15 shows plasma TREM2 levels are strongly associated with NAFLD progression. Distribution of subjects according to NASH Clinical Research Network histologic scoring system for (A) steatosis, (B) hepatocellular ballooning, and (C) lobular inflammation. (D) Logistic regression analysis for the prediction of NASH in the validation cohort (n=170) patients with biopsy-proven NAFLD. Abbreviations: HbA1c, hemoglobin A1c; BMI, body mass index; ALT, alanine aminotransferase; AST, aspartate aminotransferase; OR, odds ratio.

FIG. 16 shows association of plasma TREM2 with fibrosis. (A) Distribution of subjects according to Kleiner fibrosis stages (F0 (no fibrosis), F1 (perisinusoidal or periportal), F2 (perisinusoidal and portal/periportal), F3 (bridging fibrosis), and F4 (cirrhosis)). (B) Diagnostic accuracy of plasma TREM2 between minimal (F0-2 stages) and advanced fibrosis (F3-4 stages) patients. Correlation between plasma TREM2 levels and plasma AST levels (C), FIB-4 (D), and NFS (E) in patients with biopsy-proven NAFLD.

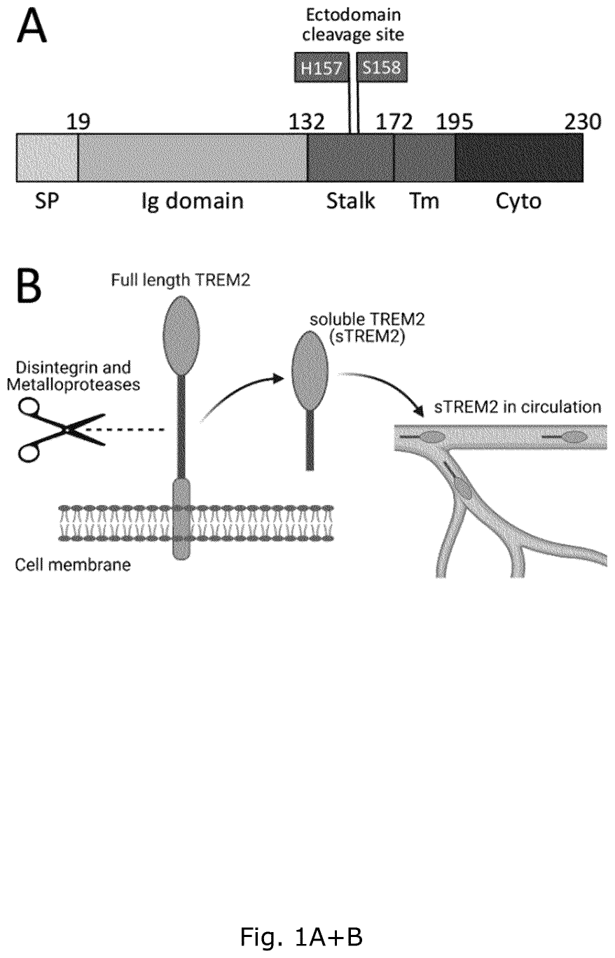

### DETAILED DESCRIPTION OF THE INVENTION

### Definitions

Prior to discussing the present invention in further details, the following terms and conventions will first be defined:

### Aspartate Transaminase to Platelet Ratio Index (APRI)

“APRI” as used herein refers to a non-invasive tool for the assessment of liver fibrosis. Based on aspartate aminotransferase and platelets in the body, the APRI score can be measured and used to determine the level of fibrosis in the liver. Three groups are used herein to describe the level of fibrosis: <0.5 equals no fibrosis, 0.5-0.98 equals mild fibrosis and >0.98 equals advanced fibrosis.

### Expression Level

The “expression level” or “level” as used herein refers to the absolute or relative amount of protein in a given sample. Thus, the expression level refers to the amount of protein in a sample. The expression level is usually detected using conventional detection methods.

The “expression level” or “level” as used herein can also refer to the absolute or relative count of gene in a given sample. Thus, the expression level refers to the count of gene in a sample. The expression level is usually detected using conventional detection methods.

In a preferred embodiment, the expression levels refer to a concentration of protein.

In another preferred embodiment, the expression level refers to the total protein level of the protein in question in a blood sample.

### FibroScan Scores

“FibroScan scores” as used herein refers to a tool for measuring fibrosis and steatosis in the liver by using ultrasound. The amount of fibrosis are divided into 4 stages (F0-F4) and the steatosis are divided into three (S1-S3).

F0/1: No or mild fibrosis, F2: moderate fibrosis, F3: severe fibrosis, F4: advanced fibrosis. S1: 11-33% of the liver affected, S2: 34-66% of the liver affected, S3: above 67% of the liver affected.

### Fibrosis 4 Index (FIB-4)

“FIB-4” as used herein refers to a method for measuring liver fibrosis. The method is an extended version of APRI where age and alanine aminotransferase are added to the calculation. Three categories are used herein: mild fibrosis (<1.45), moderate fibrosis (1.45-3.25), and advanced fibrosis (≥3.25)

### Hepatocellular Ballooning

Hepatocellular ballooning is a key finding in NASH. In histopathology, other terms are “ballooning degeneration”, “ballooning degeneration of hepatocytes”. It is conventionally defined by hemotoxylin and eosin (H&E) staining showing enlarged cells with rarefied cytoplasm and recently by changes in the cytoskeleton. Fat droplets are emerging as important organelles in cell metabolism.

Hepatocellular ballooning can be divided into different grades describing the severity. Grade 0, no ballooning; grade 1, few ballooning; and grade 2, many ballooning.

### Hepatocellular Fibrosis

“hepatocellular fibrosis” as used herein, refers to an overly exuberant wound healing in which excessive connective tissue build up in the liver. It results from chronic liver injury.

### Kleiner Fibrosis Score

The Kleiner Fibrosis score is a histological score for grading liver fibrosis. It ranges from 0 to 4, where grade zero represents no fibrosis, grade 1 is periportal OR perisinusoidal fibrosis, grade 2 is periportal AND perisinusoidal fibrosis, grade 3 represents bridging fibrosis (extending from central vein to portal triad), and grade 4 is liver cirrhosis.

### Lobular Inflammation

Lobular inflammation refers to the presence of inflammatory cell infiltrate in the hepatic lobules. The hepatic lobulus is the histological unit located between a central vein and the portal triad. The lobulus is divided into zones each representing arears with distinct hepatocyte functions. Inflammatory cells include Kupffer cells, eosinophiles, lymphocytes and neutrophiles.

### NAFLD

Non-alcoholic fatty liver disease (NAFLD), also known as “metabolic (dysfunction) associated fatty liver disease (MAFLD)”, is excessive fat build-up in the liver without another clear cause such as alcohol use. There are two types; non-alcoholic fatty liver (NAFL) and non-alcoholic steatohepatitis (NASH), with the latter also including liver inflammation. Non-alcoholic fatty liver disease is less dangerous than NASH and does not necessarily progress to NASH or liver cirrhosis. When NAFLD does progress to NASH, it may eventually lead to complications such as cirrhosis, liver cancer, liver failure, or cardiovascular disease.

### NAFLD Activity Scores (NAS)

“NAS” as used herein, refers to a scoring system for grading NAFLD. The scoring includes scoring of Steatosis (0-3), lobular inflammation (0-3), hepatocellular ballooning (0-2) and fibrosis (0-4). Unweighted summation of these forms NAS. An increase in number is equal to an increase in severity.

### NASH

Non-alcoholic steatohepatitis (NASH) is defined, as lipid accumulation with evidence of cellular damage, inflammation, and different degrees of scarring or fibrosis. NASH has been shown to be present in more than 25% of severely obese patients, 40% of whom have advanced stages of fibrosis.

### Reference Level

In the context of the present invention, the term “reference level” relates to a standard in relation to a quantity, which other values or characteristics can be compared to.

In one embodiment of the present invention, it is possible to determine a reference level by investigating the sTREM2 levels in blood samples from healthy subjects. By applying different statistical means, such as multivariate analysis, one or more reference levels can be calculated.

Based on these results, a cut-off may be obtained that shows the relationship between the level(s) detected and patients at risk. The cut-off can thereby be used e.g. to determine the sTREM2 levels, which for instance corresponds to an increased risk of having or developing NASH, preferably having NASH.

### Risk Assessment

The present inventors have successfully developed a new method to predict the risk of a subject having or developing NASH. To determine whether a patient has an increased risk of having NASH, a cut-off (reference level) must be established. This cut-off may be established by the laboratory, the physician or on a case-by-case basis for each patient.

The cut-off level could be established using a number of methods, including: multivariate statistical tests (such as partial least squares discriminant analysis (PLS-DA), random forest, support vector machine, etc.), percentiles, mean plus or minus standard deviation(s); median value; fold changes.

The multivariate discriminant analysis and other risk assessments can be performed on the free or commercially available computer statistical packages (SAS, SPSS, Matlab, R, etc.) or other statistical software packages or screening software known to those skilled in the art.

As obvious to one skilled in the art, in any of the embodiments discussed above, changing the risk cut-off level could change the results of the discriminant analysis for each subject.

Statistics enables evaluation of the significance of each level. Commonly used statistical tests applied to a data set include t-test, f-test or even more advanced tests and methods of comparing data. Using such a test or method enables the determination of whether two or more samples are significantly different or not.

The significance may be determined by the standard statistical methodology known by the person skilled in the art.

The chosen reference level may be changed depending on the mammal/subject for which the test is applied.

Preferably, the subject according to the invention is a human subject, such as a subject considered at risk of having NASH.

The chosen reference level may be changed if desired to give a different specificity or sensitivity as known in the art. Sensitivity and specificity are widely used statistics to describe and quantify how good and reliable a biomarker or a diagnostic test is. Sensitivity evaluates how good a biomarker or a diagnostic test is at detecting a disease, while specificity estimates how likely an individual (i.e. control, patient without disease) can be correctly identified as not at risk. Several terms are used along with the description of sensitivity and specificity; true positives (TP), true negatives (TN), false negatives (FN) and false positives (FP). If a disease is proven to be present in a sick patient, the result of the diagnostic test is considered to be TP. If a disease is not present in an individual (i.e. control, patient without disease), and the diagnostic test confirms the absence of disease, the test result is TN. If the diagnostic test indicates the presence of disease in an individual with no such disease, the test result is FP. Finally, if the diagnostic test indicates no presence of disease in a patient with disease, the test result is FN.

### Sensitivity

As used herein the sensitivity refers to the measures of the proportion of actual positives which are correctly identified as such, i.e. the percentage of mammals or people having a risk of having or developing NASH above normal who are identified as having risk of having or developing NASH above normal. Usually the sensitivity of a test can be described as the proportion of true positives of the total number with the target disorder i.e. a risk of developing NASH above normal. All patients with the target disorder are the sum of (detected) true positives (TP) and (undetected) false negatives (FN).

### Specificity

As used herein the specificity refers to measures of the proportion of negatives which are correctly identified—i.e. the percentage of mammal with non-increased risk of developing NASH that are identified as not having a risk of developing NASH above normal. The ideal diagnostic test is a test that has 100% specificity, i.e. only detects subjects with a risk of having developing NASH above normal and therefore no false positive results, and 100% sensitivity, i.e. detects all subjects with a risk of having or developing NASH above normal and therefore no false negative results.

For any test, there is usually a trade-off between each measure. For example, in a manufacturing setting in which one is testing for faults, one may be willing to risk discarding functioning components (low specificity), in order to increase the chance of identifying nearly all faulty components (high sensitivity). This trade-off can be represented graphically using a ROC curve.

Selecting a sensitivity and specificity it is possible to obtain the optimal outcome in a detection method. In determining the discriminating value distinguishing mammals having a risk of having or developing NASH above normal, the person skilled in the art has to predetermine the level of specificity. The ideal diagnostic test is a test that has 100% sensitivity, i.e. only detects mammals with a risk above normal of having developing NASH and therefore no false positive results, and 100% specificity, and i.e. detects all mammals with a non-increased risk of having or developing NASH and therefore no false negative results. However, due to biological diversity no method can be expected to have 100% sensitive without including a substantial number of false negative results.

The chosen specificity determines the percentage of false positive cases that can be accepted in a given study/population and by a given institution. By decreasing specificity an increase in sensitivity is achieved. One example is a specificity of 95% that will result in a 5% rate of false positive cases. With a given prevalence of 1% of e.g. a risk below normal for developing NASH in a screening population, a 95% specificity means that 5 individuals will undergo further physical examination in order to detect one (1) subject with risk above normal for developing NASH if the sensitivity of the test is 100%.

As will be generally understood by those skilled in the art, methods for screening for NASH are processes of decision making and therefore the chosen specificity and sensitivity depends on what is considered to be the optimal outcome by a given institution/clinical personnel.

### Staging

Staging as used herein describes different progression stages of NASH. A stage could e.g. be determined as a sTREM2 level above or below a certain threshold level or it could be a sTREM2 level between two thresholds if more than two stages are included in the determination.

The method according to the invention ca be combined with other diagnostic methods and biomarkers. In one embodiment, the diagnostic method and biomarker can be selected from the list: Kleiner fibrosis score, FibroScan, Aspartate transaminase, Aspartate, transaminase to platelet ratio index, CD163, TIMP1, TIMP2, MMP2, MFAP4, BMI, Sex, and Age.

### Steatosis

Steatosis, also called fatty change, is abnormal retention of fat (lipids) within a cell or organ. Steatosis most often affects the liver—the primary organ of lipid metabolism—where the condition is commonly referred to as fatty liver disease. Fat accumulation in the liver alone (steatosis) without inflammation, ballooning or fibrosis is in the benign spectrum of NAFLD, sometimes only referred to as NAFL. NASH is a malign manifestation caused by the added inflammation, ballooning and/or fibrosis. Steatosis can also occur in other organs, including the kidneys, heart, and muscle. When the term is not further specified (as, for example, in ‘cardiac steatosis’), it is assumed to refer to the liver. In the present context, steatosis is preferably liver steatosis. The severity of steatosis can be divided into grades stages where grade 0 is <5%, grade 1 is 5-33%, grade 2 is 33-67% and grade 3 is >67%. Grade 0 is described as clinically insignificant steatosis whereas grade 1-3 is described as moderate to severe steatosis.

### Steatosis, Inflammation Activity and Fibrosis (SAF) Scores

The SAF score is a histological score indicating the severity of NAFLD and unlike the NAS score it also includes fibrosis stage. Scoring of steatosis and inflammation activity is performed on HE stained hepatic tissue while fibrosis is staged in liver tissue stained with Sirius red. Steatosis is semi-quantitatively graded from 0 to 3 where zero was given if less than 5% of hepatocytes contained lipid droplets, 1 for 5 to 33%, 2 for 34 to 66% and 3 for more than 67%. The inflammation activity score can range from 0 to 4 and is based on a grading of ballooning from 0 to 2 and lobular inflammation from 0 to 2. The diagnosis if NASH cannot be given unless steatosis AND ballooning AND lobular inflammation are present (see SAF algorithm below). Fibrosis was graded from 0 to 4 as indicated by the Kleiner fibrosis score. SAF score is proposed annotated as in examples: S3A2F2 for a patient with steatosis in 34-66% of hepatocytes, moderate activity and grade 2 fibrosis.

### Subject

Reference to “subject” or an “individual” includes a human or non-human species of mammals including primates, livestock animals (e.g. sheep, cows, pigs, horses, donkey, goats), laboratory test animals (e.g. mice, rats, rabbits, guinea pigs, hamsters) and companion animals (e.g. dogs, cats). The present invention has applicability, therefore, in human medicine as well as having livestock and veterinary and wildlife applications. In a preferred embodiment, the mammal is a human.

Said subject is not diagnosed with a neurodegenerative disease, such as Alzheimer's disease (AD).

### TREM2

“Triggering receptor expressed on myeloid cells 2” or “TREM2” or “TREM-2” is a protein that in humans is encoded by the TREM2 gene. TREM2 is part of the TREM protein class of cell surface receptors of the Ig superfamily that have been shown to mediate multiple pathophysiological processes in various diseases. The TREM2 sequence is defined in Uniprot by accession number Q9NZC2.

Ectodomain of TREM2 is cleaved by disintegrin and metalloproteases including ADAM10 and ADAM17 at His157-Ser158 to generate a soluble form of TREM2 also known as sTREM2.

The invention will now be described in further details.

### Method for Determining the Risk of a Subject for Having or Developing NASH or Staging NASH

As also outlines above, the present invention relates to the use of sTREM2 as a biomarker for NASH, and/or a method for determine the effect of treatment in NASH patient based on the blood levels of sTREM2 in a subject.

Thus, a first aspect of the invention relates to a method for determining the risk of a subject having or developing NASH, the method comprising
A) determining in a blood sample from a subject, the level of soluble TREM2 (sTREM2);
B) comparing said determined level to a reference level; and
   determining that said subject is at risk of having or developing NASH, if said level is above said reference level, or
   determining that said subject is not at risk of having or developing NASH if said level is equal to or below said reference level.

Preferably, the method is for determining the risk of a subject having NASH.

In one embodiment the method is for determining the risk of a subject for having or developing NASH-associated liver inflammation and/or NASH-associated hepatocyte ballooning and/or NASH-associated Fibrosis. In a preferred embodiment, the method is for determining the risk of a subject for having NASH-associated liver inflammation and/or NASH-associated hepatocyte ballooning and/or NASH-associated Fibrosis

In another embodiment, method according to the invention is combined with one or more diagnostic methods, biomarkers or risk factors selected from the list: Kleiner fibrosis score, FibroScan, Aspartate transaminase, Aspartate transaminase to platelet ratio index, CD163, TIMP1, TIMP2, MMP2, MFAP4, BMI, Sex and Age.

The blood sample wherein sTREM2 is measured is obtained from a subject.

The “subject” as described herein comprises humans of all ages, other primates (e.g., cynomolgus monkeys, rhesus monkeys); mammals in general, including commercially relevant mammals such as cattle, pigs, horses, sheep, goats, mink, ferrets, hamsters, cats, dogs; and/or birds. Preferred subjects are humans.

Thus, in an embodiment of the present invention, the subject is selected from the group consisting of; humans of all ages, other primates (e.g., cynomolgus monkeys, rhesus monkeys); mammals in general, including commercially relevant mammals, such as cattle, pigs, horses, sheep, goats, mink, ferrets, hamsters, cats and dogs, as well as birds.

In a preferred embodiment, the subject is a mammal, preferably a human.

The subjects can be grouped according to the Body mass index (BMI). BMI is a value derived from the mass (weight) and height of the subject. BMI is according to the present invention defined as body mass divided by the square of the body height and is expressed in units of kg/m².

In relation to the present invention BMI value below 18.5 indicates a underweight subject, BMI values of 18.5-25 indicate a normal weight subject, BMI value 25-30 indicate an overweight subject and BMI values above 30 indicate an obese subject.

In one embodiment, subject has a BMI above 25, preferably, the subject has a BMI above 30, more preferably above 35, even more preferably above 40 and even more preferably, the subject has a BMI above 45.

The level of sTREM2 can be combined with other parameters to increase the accuracy of the diagnosis. One method that can be applied together with the measurement of sTREM2 levels is the measurement of liver stiffness. This is a non-invasive method based on ultrasound, which can determine late-stage fibrosis.

In one embodiment, the level of sTREM2 can be combined with other parameters, which may be any diagnostic methods, biomarkers or risk factors as described herein.

In another embodiment, the method according to the invention can be combined with Kleiner fibrosis score, FibroScan, Aspartate transaminase, Aspartate transaminase to platelet ratio index, CD163, TIMP1, TIMP2, MMP2, MFAP4, BMI, Sex and Age.

In a further embodiment, the method is combined with non-invasive determination of liver stiffness, such as by transient elastiometry (TE).

As describes above, the method comprises a step wherein a “reference level” is used.

In one embodiment, the reference level is determined in a sample obtained from a healthy subject.

In another embodiment, the reference level is determined as an average of measurements in samples obtained from a group of healthy subjects.

In yet another embodiment, the reference level is determined in a corresponding sample from the same subject, wherein said corresponding sample has been obtained at a previous time point.

The skilled person may apply different reference levels depending on the desired specificity and sensitivity. Thus, in one embodiment, the reference level of sTREM2 is at least 28 ng/ml, such as at least 30 ng/ml, such as at least 33 ng/ml, such as at least 35 ng/ml, such as at least 37 ng/ml, such as at least 40 ng/ml or in the range of 28-50 ng/ml, such as in the range 28-45 ng/ml or in the range 30-40 ng/ml, such as in the range 33-37 ng/ml.

In another embodiment, the reference level of sTREM2 blood plasma is at least 28 ng/ml, such as at least 30 ng/ml, such as at least 33 ng/ml, such as at least 35 ng/ml, such as at least 37 ng/ml, such as at least 40 ng/ml or in the range of 28-50 ng/ml, such as in the range 28-45 ng/ml or in the range 30-40 ng/ml, such as in the range 33-37 ng/ml.

In a further embodiment, the reference level of sTREM2 blood serum is at least 28 ng/ml, such as at least 30 ng/ml, such as at least 33 ng/ml, such as at least 35 ng/ml, such as at least 37 ng/ml, such as at least 40 ng/ml or in the range of 28-50 ng/ml, such as in the range 28-45 ng/ml or in the range 30-40 ng/ml, such as in the range 33-37 ng/ml.

As outlined in example 8, a cut-off at 38 ng/ml was optimal to rule-out (exclude) NASH patients with a sensitivity of 90% (95% CI: 81-96%) and specificity 52%; whereas plasma TREM2 level of 65 ng/ml was the optimal rule-in (diagnose) cut-off with a specificity of 89% (95% CI: 81-94%) and sensitivity 54%.

Thus, in a yet a further embodiment, the reference level of sTREM2 (blood serum or blood plasma, is at least 28 ng/ml, such as at least 30 ng/ml, preferably such as at least 33 ng/ml, more preferably such as at least 35 ng/ml, such as at least 37 ng/ml, such as at least 40 ng/ml or in the range of 28-100 ng/ml, preferably such as in the range 30-80 ng/ml or in the range 30-70 ng/ml.

The level of sTREM2 is, as outlined above, determined in a sample.

The samples may be obtained as blood samples according to the invention.

Thus, in one embodiment the sample is a blood sample.

In preferred embodiment, the sample is a blood plasma sample.

In another preferred embodiment, the sample is a blood serum sample.

When the level of sTREM2 is measured in a sample according to the invention, the level is relative to the sample size and thus, presented as a concentration.

Thus, in one embodiment, the level of sTREM2 is the concentration of sTREM2.

sTREM2 levels may be determined in different ways. Thus, in one embodiment, the level of sTREM2 is determined at the protein level.

In another embodiment, the protein level is performed using a method selected from the group comprising immunohistochemistry, immunocytochemistry, immunoturbidimetry, FACS, ImageStream, Western Blotting, ELISA, Luminex, Multiplex, Immunoblotting, TRF-assays, immunochromatographic lateral flow assays, Enzyme Multiplied Immunoassay Techniques, RAST test, Radioimmunoassays, immunofluorescence and immunological dry stick assays, such as a lateral flow assay.

In a preferred embodiment, the level of sTREM2 is determined by ELISA, multiplexing or immunoturbidimetry.

### Monitoring the Development

As outlined above, the method according to the invention can be used to determine the risk of having or developing NASH at a given time point. However, the method of the invention might also find use to monitor the development of NASH in a subject. Thus, an aspect of the invention relates to a method for monitoring the development of NASH in a subject, the method comprising
determining a first level of sTREM2 in a first blood sample from the subject;
determining a second level of sTREM2 in a second blood sample from the subject, wherein the second sample has been obtained at a later time point than the first sample;
comparing corresponding levels in the first and second sample;
wherein
   a higher sTREM2 level in the second sample compared to the first sample is indicative of a worsened in NASH;
   an equal or lower sTREM2 level in the second sample compared to the first sample is indicative of unchanged or improvement in NASH.

In one embodiment, the first and second sample is obtained from the same subject at two separate time points.

In another embodiment, the second sample can be followed by a third and fourth sample obtained separately at later time points.

In a further embodiment, a treatment against NASH has taken place between the sampling of the first and second sample.

### Effect of Treatment

It may also be advantageous to be able to monitor if a treatment for NASH is efficient. Thus, an aspect of the present invention relates to a method for determining the effect of a treatment protocol against NASH for a subject, the method comprising
determining a first level of sTREM2 in a first blood sample from the subject;
determining a second level of sTREM2 in a second blood sample from the subject, wherein the second sample has been obtained at a later time point than the first sample;
wherein the treatment protocol has been initiated or completed before the sampling of the first sample or initiated, continued or completed between the sampling of the first and the second sample,
comparing sTREM2 levels in the first sample and the second sample; wherein
   a sTREM2 level in the second sample below or equal to the sTREM2 level in the first sample is indicative of the treatment protocol being efficient against NASH or,
   a sTREM2 level in the second sample above the sTREM2 level in the first sample is indicative of the treatment protocol not being efficient against NASH.

In one embodiment, treatment is treatment selected from the group consisting of CENICRIVIROC and tropifexor (in combination or separately), RESMETIROM, OCALIVA, obeticolic acid, ELAFIBRANOR, ARAMCHOL, IMM124E, SEMAGLUTIDE, liraglutide, LANIFIBRANOR, SELADELPAR, BELAPECTIN, PXL_065, MSDC_0602, ALDAFERMIN, VK2809, EDP_305, HTD1801, PF_05221304, TIPELUKAST, TROPIFEXOR, DF102, LMB763, NITAZOXANIDE, TESAMORELIN, SELADELPAR, TERN_101, LAZAROTIDE, BMS986036, SAROGLITAZAR, AKR001, CRV431, GRI_0621, EYP0010, BMS_986171, ISOSABUTATE, PF_06835919, PF_06865571, NALMEFENE, LIK066, BIO89_100, NAMODENOSON, MT_3995, PERMAFIBRATE, PXL770, GEMCABENE, FORALUMAB, SGM_1019, KBP_042, HEPASTEM, CER_209, DUR928, SOTAGLIFLOZIN, ELOBIXIBAT, SAR425899, NGM313, NAMACIZUMAB, TERN_201, LPCN_1144, ND_L02_s0201, RTU_1096, IONIS_DGAT2Rx, BEZAFIBRATE, INT_767, NP160, NEULIV, NP135, BFKB8488A, NC_001, VK0214, HM15211, CM_101, AZD2693, NV556, SP_1373, RLBN1127, RYI_018, NVP022, VPR_423, CB4209-CB4211, and GKT_137831.

In a preferred embodiment, treatment is treatment selected from the group consisting of CENICRIVIROC, RESMETIROM, obeticolic acid, ARAMCHOL, IMM124E, SEMAGLUTIDE, Liraglutide, LANIFIBRANOR, SELADELPAR, PXL_065 and SDC_0602.

In a more preferred embodiment, treatment is treatment selected from the group consisting of RESMETIROM, ELAFIBRANOR, ARAMCHOL, SEMAGLUTIDE, and LANIFIBRANOR.

Patients can in addition be treated by different surgical procedures.

Thus, in one embodiment, the treatment protocol is a surgical procedure.

In another embodiment, the treatment is a surgical procedure selected from the group consisting of Bariatric surgery, Roux-en-Y gastric bypass, Gastric sleeve operation and Adjustable gastric band.

In yet another embodiment, the treatment is a change of lifestyle, such as change of diet, exercise, alcohol consumption, no smoking and/or reduction of smoking.

Most patients diagnosed with NASH are overweight or obese. Thus, in one embodiment, said treatment is selected from the group of pharmaceutical compounds approved for the treatment of weight loss or hepatic metabolic optimization, such as selected from the group consisting of metformin, statins, GLP-1 analogues such as semaglutide or liraglutide; and also include compounds to be approved in the future e.g., anti-fibrotic compounds.

### Use of Blood Sample Levels of sTREM2 From a Subject as a Biomarker

The present invention can also be used as a biomarker for determining if a subject has an increased risk of having or developing NASH or staging of NASH by determining the level of sTREM2 in a sample ex vivo. Thus, an aspect of the present invention relates to the use of blood sample levels of sTREM2 from a subject as a biomarker for the risk of said subject having or developing NASH or staging NASH for said subject, preferably having NASH.

In an embodiment, the use is for determining for said subject the risk of having NASH.

In one embodiment, sTREM2 levels are determined in a plasma sample or a serum sample.

In another embodiment, the level of sTREM2 is determined ex vivo.

In one embodiment, if said subject is determined to be at risk of having or developing NASH a treatment against NASH is initiated.

In another embodiment, treatment is treatment selected from the group consisting of CENICRIVIROC and tropifexor (separately or in combination), RESMETIROM, OCALIVA, obeticolic acid, ELAFIBRANOR, ARAMCHOL, IMM124E, SEMAGLUTIDE, liraglutide, LANIFIBRANOR, SELADELPAR, BELAPECTIN, PXL_065, MSDC_0602, ALDAFERMIN, VK2809, EDP_305, HTD1801, PF_05221304, TIPELUKAST, TROPIFEXOR, DF102, LMB763, NITAZOXANIDE, TESAMORELIN, SELADELPAR, TERN_101, LAZAROTIDE, BMS986036, SAROGLITAZAR, AKR001, CRV431, GRI_0621, EYP0010, BMS_986171, ISOSABUTATE, PF_06835919, PF_06865571, NALMEFENE, LIK066, BIO89_100, NAMODENOSON, MT_3995, PERMAFIBRATE, PXL770, GEMCABENE, FORALUMAB, SGM_1019, KBP_042, HEPASTEM, CER_209, DUR928, SOTAGLIFLOZIN, ELOBIXIBAT, SAR425899, NGM313, NAMACIZUMAB, TERN_201, LPCN_1144, ND_L02_s0201, RTU_1096, IONIS_DGAT2Rx, BEZAFIBRATE, INT_767, NP160, NEULIV, NP135, BFKB8488A, NC_001, VK0214, HM15211, CM_101, AZD2693, NV556, SP_1373, RLBN1127, RYI_018, NVP022, VPR_423, CB4209-CB4211, and GKT_137831.

In a preferred embodiment, treatment is treatment selected from the group consisting of CENICRIVIROC, RESMETIROM, obeticolic acid, ARAMCHOL, IMM124E, SEMAGLUTIDE, Liraglutide, LANIFIBRANOR, SELADELPAR, PXL_065, and MSDC_0602.

In a more preferred embodiment, the treatment is selected from the group consisting RESMETIROM, ELAFIBRANOR, ARAMCHOL, SEMAGLUTIDE, and LANIFIBRANOR.

Patients can in addition be treated by different surgical procedures.

In one embodiment, the treatment is a surgical procedure selected from the group consisting of Bariatric surgery, Roux-en-Y gastric bypass, Gastric sleeve operation and Adjustable gastric band.

In another embodiment, the treatment is a change of lifestyle, such as change of diet, exercise, alcohol consumption, no smoking and/or reduction of smoking.

In another embodiment, said treatment is selected from the group of pharmaceutical compounds approved for treatment of weight loss or hepatic metabolic optimization: metformin, statins, GLP-1 analogues such as semaglutide or liraglutide; and also include compounds to approved in the future e.g., anti-fibrotic compounds.

### EXAMPLES

### Example 1—Materials and Methods

### Study Design and Participants

Liver and blood samples were collected from obese participants in an on-going biopsy-controlled, single-center, prospective interventional study (PROMETHEUS). Blood samples were also collected from healthy but obese participants from another prospectively conducted study (GALA-HP). The committee on health research ethics for Southern Denmark approved both studies (S-20170210; S-20160006G). PROMETHEUS is registered at ClinicalTrials.gov (NCT03535142) while GALA-HP is registered at OPEN.rsyd.dk (OP-239; Odense Patient data Explorative Network). The data used here were all cross-sectional baseline data. We included healthy controls from May 2016 to March 2018, and obese subjects from August 2018 to January 2021. From a total of 64 patients in the total cohort enrolled for the PROMETHEUS study, we selected 30 patients for TREM2 analysis based on histological NAS-Clinical Research Network (NAS-CRN): A no-NASH group of 10 with a NAS score of zero, and a NASH-group of 20 with ballooning and NAS score 3-8. From 154 healthy participants enrolled in GALA-HP study, we selected 10 to match for age and gender to the obese cases, making the total number selected for this study at n=40 (patient characteristics are presented in Table 1).

TABLE 1
Clinicopathological characteristics.

| | Total | No NASH | NASH | |
|---|---|---|---|---|
| Clinical features | (n = 40) | (n = 20) | (n = 20) | P-value |
| Age | 50 + 18.5 | 46 + 15.5 | 50 + 20 | 0.55 |
| Gender (Female/Male) | 27/13 | 14/6 | 13/7 | 0.75 |
| BMI (kg/m 2) | 42.6 + 7.9 | 44.35 + 6.3 | 41.45 + 8.15 | 0.71 |
| Fraction with T2DM | 10/40 | 0/20 | 10/20 | <0.0001 |

Biochemical variables

| | | | | |
|---|---|---|---|---|
| ALT (U/L) | 31 + 40.5 | 18.5 + 8 | 58.5 + 40.5 | <0.0001 |
| AST (U/L) | 25 + 22 | 21 + 9 | 35.5 + 32 | 0.003 |
| Platelet (x109/L) | 251.5 + 61 | 252 + 92.5 | 251 + 70.5 | 0.11 |
| Triglycerides (mmol/L) | 1.23 + 1.02 | 1 + 0.185 | 2 + 1.16 | 0.0003 |
| Total cholesterol (mmol/L) | 4.8 + 1.05 | 4.7 + 1 | 4.8 + 1 | 0.73 |
| CRP (mg/L) | 7.6 + 9.4 | 10.5 + 7.7 | 6 + 7.8 | 0.17 |
| HOMA-IR | 4.54 + 7.7 | 2.75 + 2.22 | 10.45 + 9.15 | 0.0009 |

Histologic variables

| | | | | |
|---|---|---|---|---|
| NAFLD activity score | 1.5 + 5 | 0 | 5 + 1 | <0.0001 |
| SAF activity score | 3 + 2 | 1 + 0 | 3 + 0 | <0.0001 |
| Steatosis grade 0/1/2/3 | 20/4/12/4 | 20/0/0/0 | 0/4/12/4 | <0.0001 |
| Ballooning grade 0/1/2 | 20/18/2 | 20/0/0 | 0/18/2 | <0.0001 |
| Lobular inflammation grade 0/1/2/3 | 20/3/14/3 | 20/0/0/0 | 0/3/14/3 | <0.0001 |
| Kleiner fibrosis grade F0/F1/F2/F3 | 17/11/8/4 | 17/0/0/0 | 0/11/8/4 | <0.0001 |

Non-invasive markers of liver fibrosis

| | | | | |
|---|---|---|---|---|
| FibroScan | 7.65 + 8.95 | 3.9 + 4 | 11.9 + 13.1 | 0.0005 |
| APRI | 0.24 + 0.29 | 0.23 + 0.14 | 0.36 + 0.42 | 0.04 |
| FIB-4 | 3.11 + 3.38 | 2.32 + 2.32 | 3.86 + 2.57 | 0.09 |

All data in Table 1 are expressed in median ±interquartile range or frequency. P-value reports unpaired T-test between no-NASH and NASH groups, significance level at P<0.05. BMI: Body Mass Index, T2DM: Type 2 Diabetes Mellitus, ALT: Alanine aminotransaminase, AST: Aspartate aminotransaminase, CRP: C-reactive protein, NAFLD: Non-Alcoholic Fatty Liver Disease, SAF: Steatosis Activity Fibrosis, HOMA-IR: Homeostatic Model Assessment for Insulin Resistance, APRI: AST to Platelet Ratio Index, FIB-4: Fibrosis-4.

TABLE 2
Individual patient NAFLD scores and their
corresponding sTREM2 values (ng/ml).

| NAFLD activity score | STREM2 conc. (ng/ml) |
|---|---|
| 0 | 15.73 |
| 0 | 21.57 |
| 0 | 18.41 |
| 0 | 13.09 |
| 0 | 17.78 |
| 0 | 12.8 |
| 0 | 24.89 |
| 0 | 12.01 |
| 0 | 20.13 |
| 0 | 27.72 |
| 0 | 27.10 |
| 0 | 14.16 |
| 0 | 24.09 |
| 0 | 18.73 |
| 0 | 25.24 |
| 0 | 7.04 |
| 0 | 15.31 |
| 0 | 16.62 |
| 0 | 15.34 |
| 0 | 11.11 |
| 3 | 96.05 |
| 4 | 97.82 |
| 4 | 48.49 |
| 4 | 106.83 |
| 4 | 94.22 |
| 5 | 50.8 |
| 5 | 45.45 |
| 5 | 43.47 |
| 5 | 106.1 |
| 5 | 46.65 |
| 5 | 51.87 |
| 5 | 62.33 |
| 5 | 50.12 |
| 5 | 46.34 |
| 5 | 60.01 |
| 6 | 85.05 |
| 6 | 176.13 |
| 6 | 55.19 |
| 7 | 196.87 |
| 8 | 169.37 |

### Clinical and Laboratory Assessment

Blood and tissue samples were performed on the same day in the early hours from 8-12 PM, and all participants were in a fasting state. Blood samples were drawn by trained lab staff and processed by a single lab technician for biobanking and routine tests shortly thereafter. Routine biochemical analyses were done on a Cobas 6000 (Roche) and included aspartate transaminase (AST), alanine aminotransferase (ALT), Alkaline Phosphatase, Bilirubin, International Normalized Ratio (INR), Creatinine, Sodium, Albumin, C-Reactive Protein (CRP), Cholesterol, HDL/LDL and Triglycerides. EDTA plasma samples for biomarker analysis were stored at −80° C. until analysis.

### Liver Stiffness Measurement

An experienced nurse performed liver elastography using FibroScan (Echosens, Paris, France) to obtain a non-invasive measure for liver fibrosis. Liver stiffness measurements (LSM) were performed on the same day as, but prior to, the liver biopsies. All included subjects had a baseline liver biopsy performed regardless of LSM.

### Histological Staging of NAFLD

Percutaneous liver biopsies were obtained during a sterile procedure using a 17-G Menghini needle (Hepafix, Braun, Germany). A single experienced Pathologist evaluated all liver biopsies blinded to other results. We staged liver steatosis (0-3), ballooning (0-2) and lobular inflammation (0-3) according to the NAS. We used Kleiner score to stage liver fibrosis (F0-4).

### ELISA

TREM2, matrix metalloproteinase 2 (MMP2), tissue inhibitor matrix metalloproteinase 1 (TIMP1) or tissue inhibitor matrix metalloproteinase 2 (TIMP2) levels in plasma or serum samples were analyzed using the respective Human ELISA Kits (Abcam) according to manufacturer's instructions. Briefly, 50 μL of each standard and sample were added to appropriate wells followed by the addition of 50 μL of antibody cocktail (detector and capture antibody mixture) and incubated for 1 hr at RT with gentle shaking (400 rpm). After washing, 100 μL of 3,3′,5,5′-Tetramethylbenzidine Development Solution was added to all wells and incubated for 10 mins in dark at RT with gentle shaking (400 rpm). Approximately 100 μL of Stop Solution was added to all wells followed by measurement of absorbance at 450 nm in a UV/Vis microplate spectrophotometer (Thermo Scientific, Multiskan GO).

### Statistical Analyses

The significance between two groups was calculated using unpaired two-tailed Student t test. Group differences involving multiple subtypes in steatosis, inflammation, ballooning and fibrosis were tested by the simple One-way ANOVA test. Correlation between two groups were computed with Pearson correlation coefficient (two-tailed P-value) and represented as ‘r’. ROC curves were used to determine the specificity and sensitivity and were expressed as AUROC. The cutoff points were selected using the Youden index, which maximizes the sum of sensitivity and specificity. Individual RNAseq count tables and associated metadata (disease grade, NAS and fibrosis score) from 216 human liver biopsies were retrieved from GEO (GSE135251) and assembled into a single count table. The RNAseq data was subsequently normalized by DESeq2 and TREM2 (ENSG00000095970) expression data was extracted for further analysis. P<0.05 was considered statistically significant. Data are presented as the means+SD or median+interquartile range, as indicated. All figures were prepared and analyzed using GraphPad Prism version 9.1.0.221.

### Example 2—TREM2 Expression is Upregulated in NASH

### Aim of Study

To evaluate the clinical relevance of TREM2 in NASH patients, we performed an analysis of the publicly available RNA seq data from 216 liver biopsies isolated from NAFLD patients.

Materials and methods

See example 1.

### Results

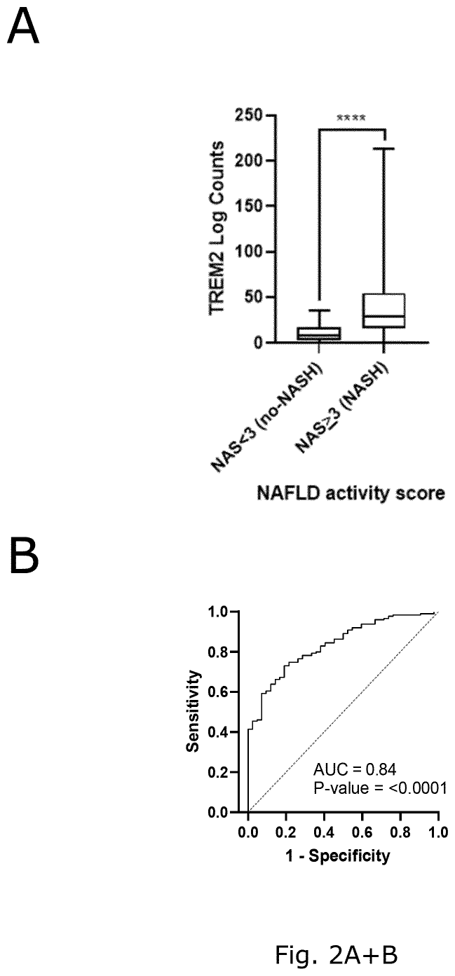

To independently validate the regulation of TREM2 gene in NASH patients, we analysed the publicly available RNA seq data from 216 liver biopsies isolated from NAFLD patients (12). In this analysis, we found the TREM2 gene to be highly differentially expressed in NASH patients as compared with no-NASH controls (FIG. 2A) with a diagnostic accuracy of AUROC 0.84 (95% CI: 0.78-0.89,P<0.0001) and a sensitivity of 73% (95% CI: 0.66-0.79) and specificity of 81% (95% CI: 0.66-0.90) (FIG. 2B).

### Conclusion

Taken together, we conclude that liver tissue-associated TREM2 is highly upregulated in NASH patients (NAS≥3).

### Example 3—Soluble TREM2 Levels can Discriminate NASH From No-NASH Patients

### Aim of Study

With the aim of exploring the utility of sTREM2 as a non-invasive biomarker to discriminate NASH patients from no-NASH controls, we performed quantitative immunoassay (ELISA) on plasma extracted from 20 NASH patients and 20 no-NASH controls.

Materials and methods

See example 1.

### Results

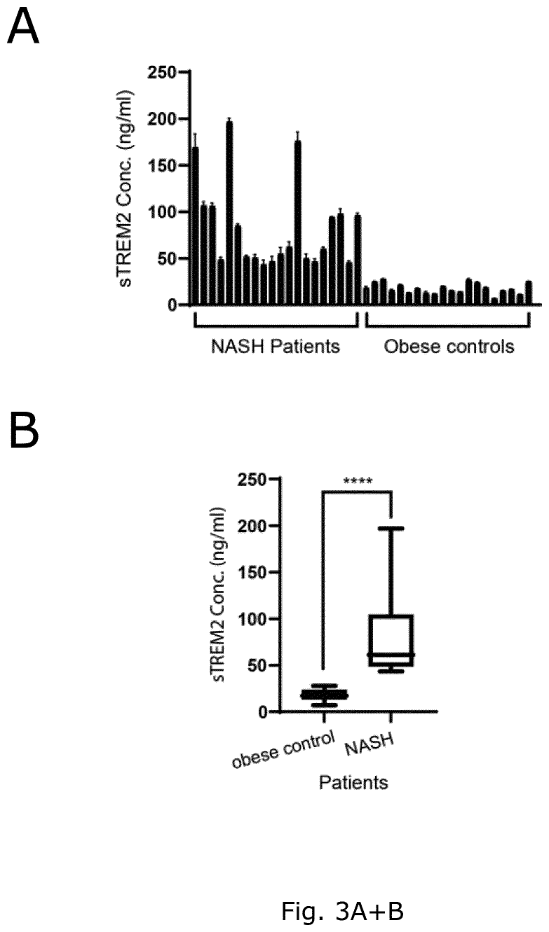

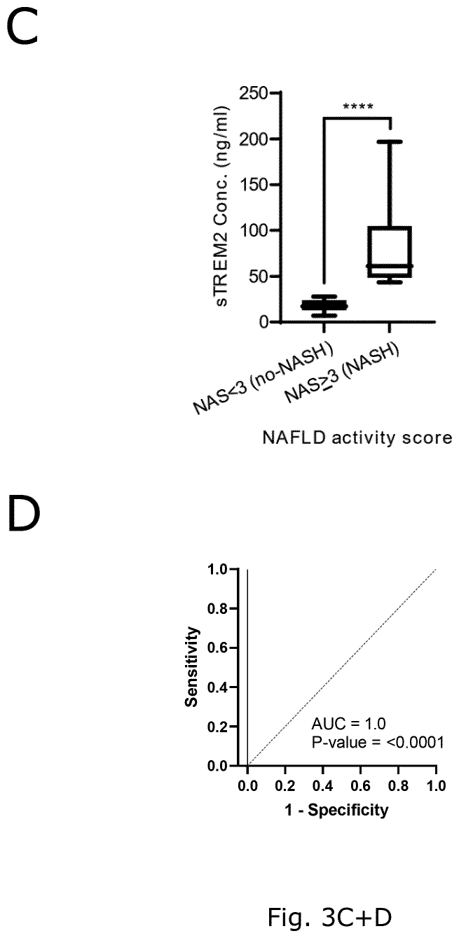

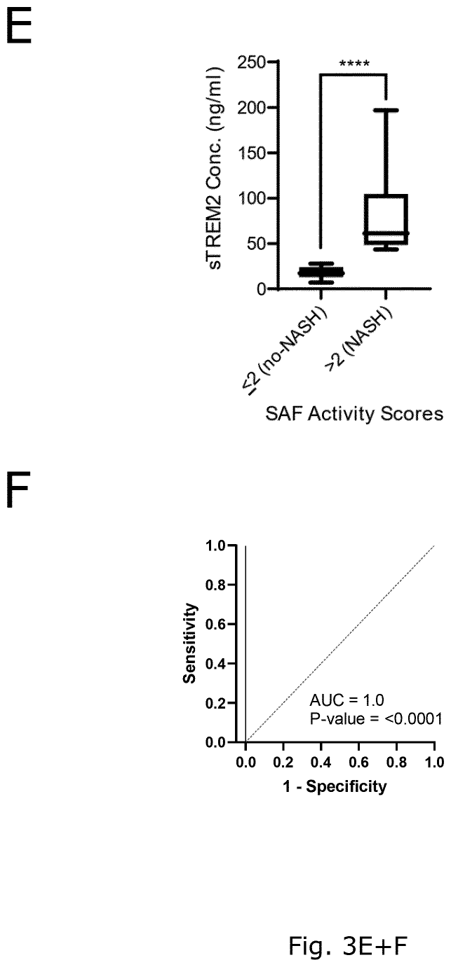

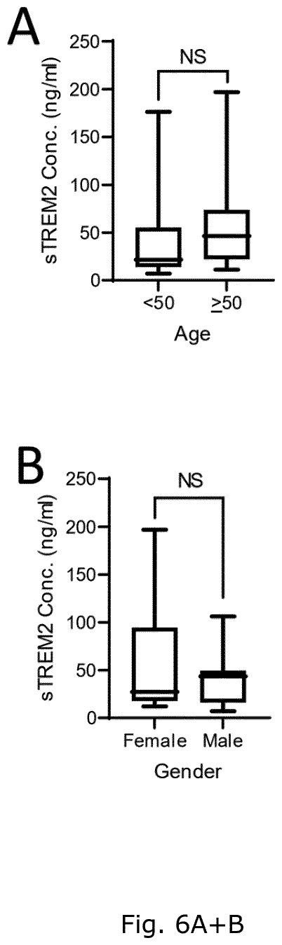

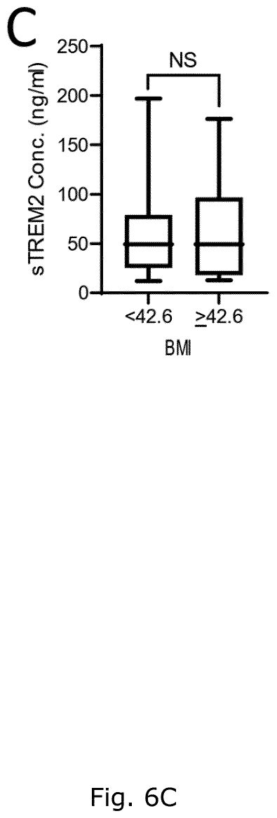

sTREM2 was detected in the plasma of all NASH patients and no-NASH controls above the lower limit of detection (78.1 pg/ml) (FIG. 3A). There was no overall difference in age, gender, or BMI (cutoff at median values from Table 1) between NASH patients and no-NASH controls (Age (50 Y), P=0.75; Gender, P=0.24; BMI (42.6 kg/m2), P=0.72) (FIG. 6A-C).

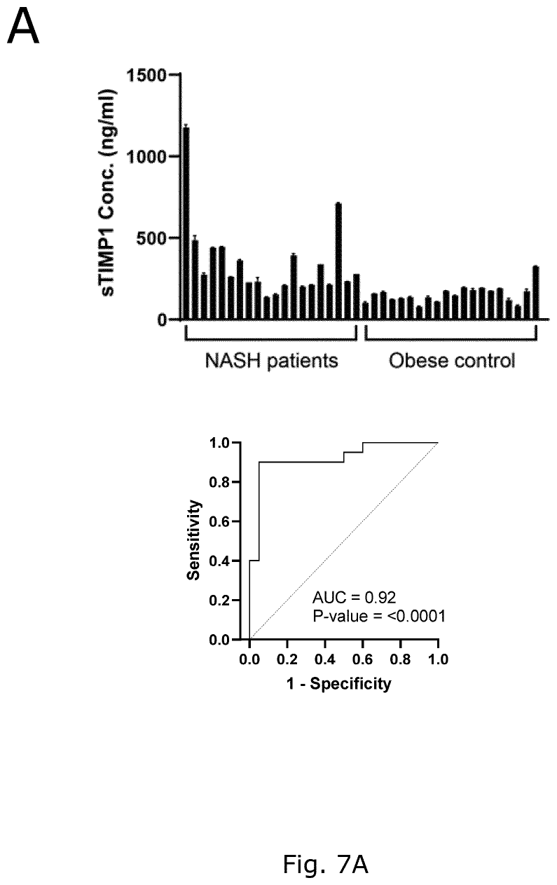

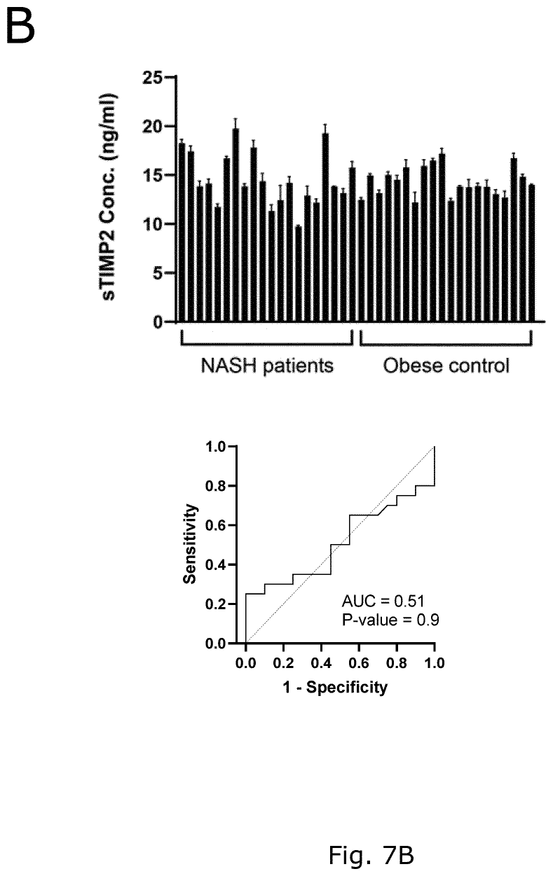

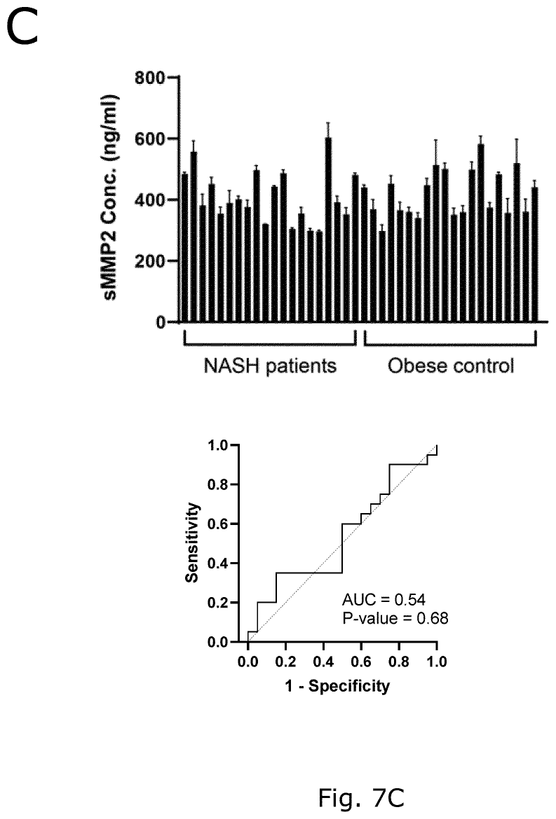

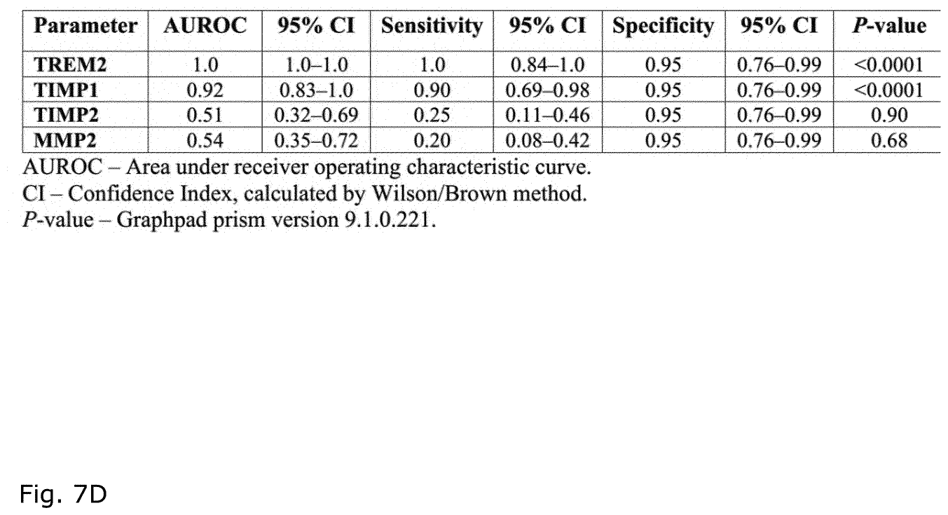

sTREM2 was increased by 4.7-fold (P<0.0001) in NASH patients as compared with no-NASH obese control groups (FIG. 3B). When patients were dichotomized based on NAFLD activity scores into two groups (cutoff at <3 (no-NASH) and ≥3 (NASH) (16), sTREM2 diagnosed NASH from no-NASH patients (P<0.0001) (FIG. 3C) with an excellent discriminatory accuracy of AUROC 1.0 (95% CI: 1.0-1.0, P<0.0001) and a sensitivity of 100% (95% CI: 0.84-1.0) and specificity of 95% (95% CI: 0.76-0.99) (FIG. 3D). Additionally, evaluation of patients based on Steatosis, Inflammation Activity and Fibrosis (SAF) scores (cut-off at ≥2 as NASH and ≤2 as no-NASH) (19) showed excellent diagnostic discrimination of NASH and no-NASH patient groups by sTREM2 (P<0.0001) (FIG. 3E) with an AUROC of 1.0 (95% CI: 1.0-1.0, P<0.0001) and a sensitivity of 100% (95% CI: 0.84-1.0) and specificity of 95% (95% CI: 0.76-0.99) (FIG. 3F). Also, sTREM2 outperformed the diagnostic accuracy of recognized NASH-associated circulating markers including TIMP1 (AUROC of 0.92, P<0.0001), TIMP2 (AUROC of 0.51, P=0.9) and MMP2 (AUROC of 0.54, P=0.68) (FIG. 7A-D).

### Conclusion

In conclusion, circulating sTREM2 levels effectively discriminate patients with elevated NASH (NAS≥3) from no-NASH patients with excellent diagnostic accuracy and also discriminated NASH patients from no-NASH using SAF activity score.

### Example 4—Plasma sTREM2 Levels are Associated With NAFLD Progression

### Aim of Study

To determine the association of sTREM2 with progressive stages of NAFLD, we adopted the NASH Clinical Research Network (NASH CRN) histologic scoring system as described by Kleiner et al (9) for steatosis (grade 0, <5%; grade 1, 5-33%; grade 2, 33-67%; and grade 3, >67%), inflammation (grade 0 no foci; grade 1, <2 foci per 20× field; grade 2, 2-4 foci per 20× field; and grade 3, >4 foci per 20× field) and ballooning (grade 0, no ballooning; grade 1, few ballooning; and grade 2, many ballooning).

### Materials and Methods

See example 1.

### Results

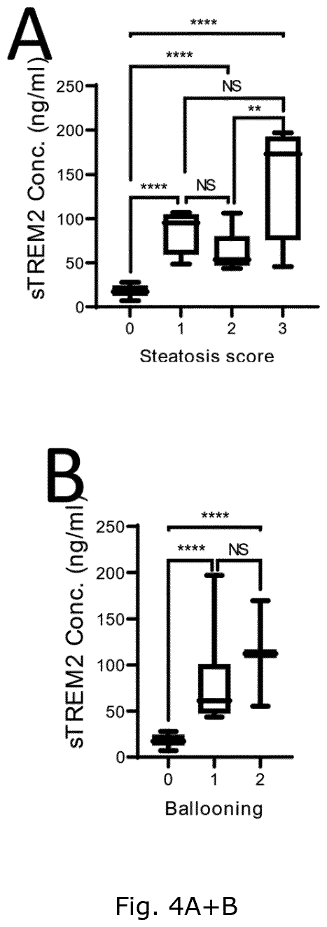

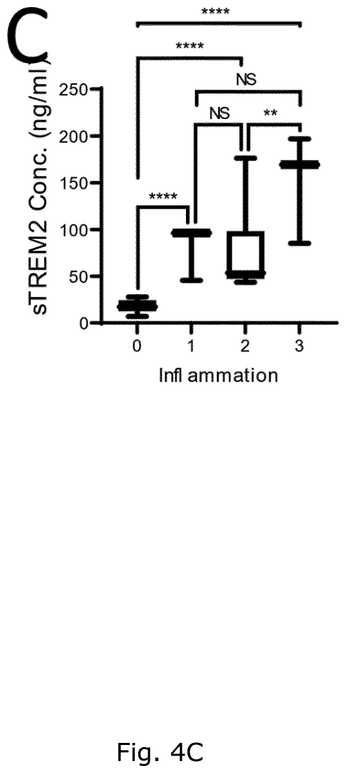

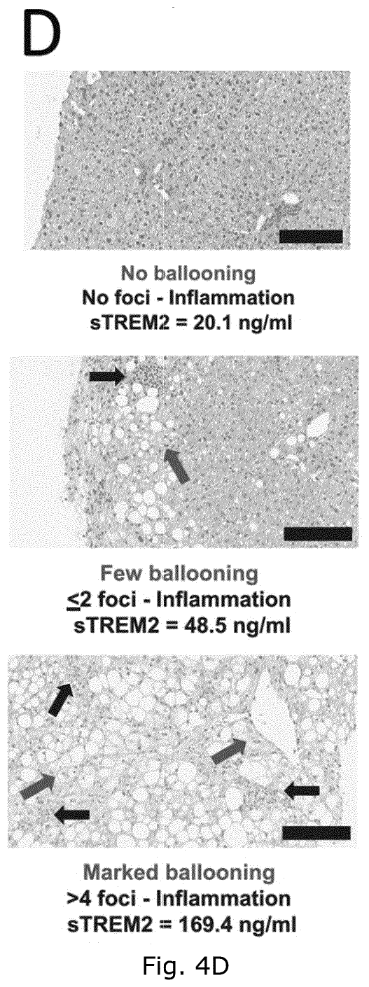

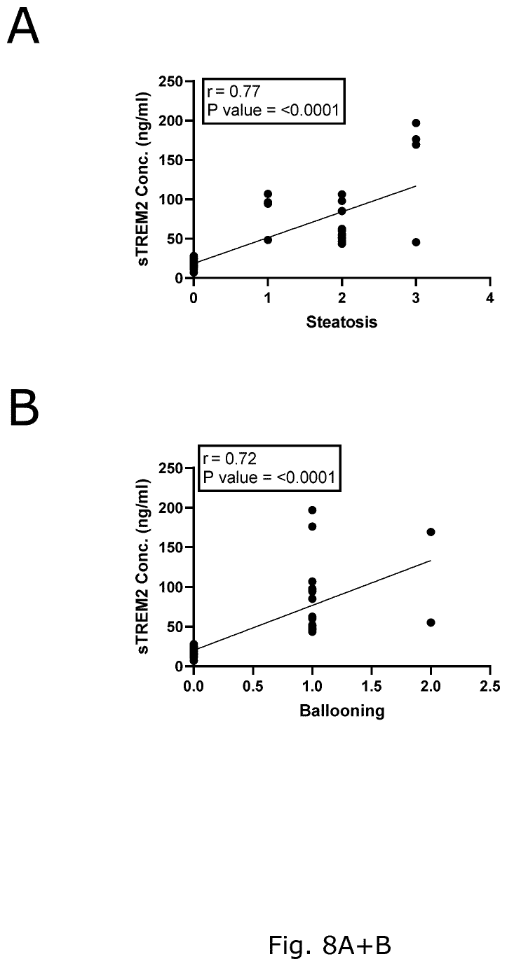

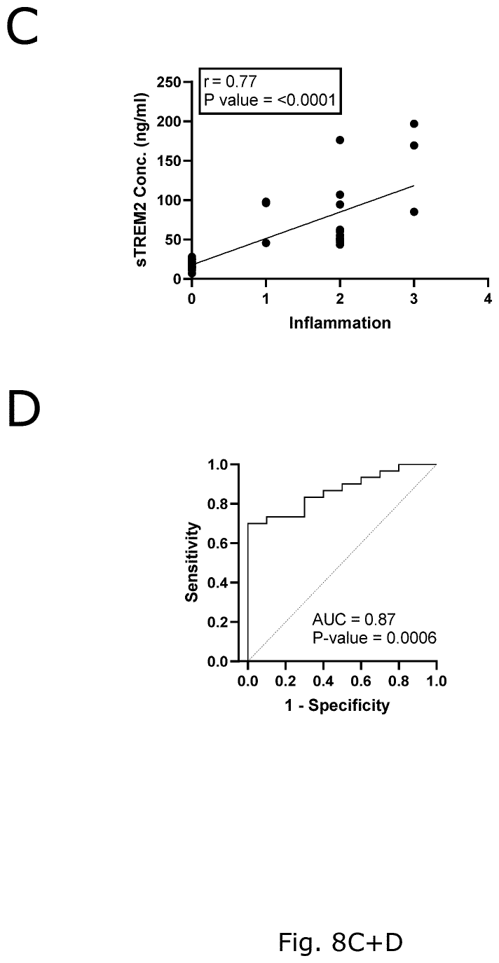

Patient (n=40) plasma sTREM2 levels correlated with steatosis (r=0.77, P<0.0001) (FIG. 8A), ballooning (r=0.72, P<0.0001) (FIG. 8B) and lobular inflammation (r=0.77, P<0.0001) (FIG. 8C). We observed significantly elevated levels of sTREM2 in patients with moderate and severe steatosis (grade 1-3) as compared to patients with clinically insignificant grade 0 steatosis (Grade 0-1, P<0.0001; Grade 0-2, P<0.0001; Grade 0-3, P<0.0001) (FIG. 4A). Likewise, patients with marked ballooning (grade 1 and 2) had elevated levels of sTREM2 as compared with patients with no ballooning degeneration (Grade 0-1, P<0.0001; Grade 0-2, P<0.0001) (FIGS. 4B and 4D). In the case of lobular inflammation, patients with moderate to severe inflammation (grade 1-3, >2 foci) had significantly higher levels of sTREM2 as compared to patients with no inflammation (grade 0-1, <2 foci, P<0.0001; Grade 0-2, P<0.0001; Grade 0-3, P<0.0001) (FIGS. 4C and 4D). We conclude that sTREM2 is strongly associated with severe liver histopathological features of steatosis, ballooning and lobular inflammation. Taking cue from this inference, we sought to determine if sTREM2 can discriminate NAFLD patients with insulin resistance having worse liver histology and rule out NAFLD patients without IR. For this, we applied the Homeostatic Model Assessment of Insulin Resistance (HOMA-IR) scores (≥2.73, NAFLD with IR; <2.73, NAFLD without IR) and expectedly, sTREM2 levels effectively differentiated NAFLD with IR patients from NAFLD without IR with an AUROC of 0.87 (95% CI: 0.76-0.98, P=0.0006) and a sensitivity of 73% (95% CI: 0.56-0.86) and specificity of 90% (95% CI: 0.6-0.99) (FIG. 8D).

### Conclusion

In conclusion, plasma sTREM2 levels correlate with steatosis, ballooning, and lobular inflammation in NASH patients.

### Example 5—Plasma sTREM2 Levels can Accurately Diagnose Advanced Liver Fibrosis

### Aim of Study

To evaluate the relevance of sTREM2 in the diagnosis of hepatic fibrosis patients, we applied multiple invasive and non-invasive fibrosis classification systems.

### Materials and Methods

See also example 1.

Patients (n=40) were categorized to F0 (no fibrosis), F1 (portal), F2 (periportal), F3 (bridging fibrosis), and F4 (cirrhosis) groups according to the Kleiner fibrosis score (16).

### Results

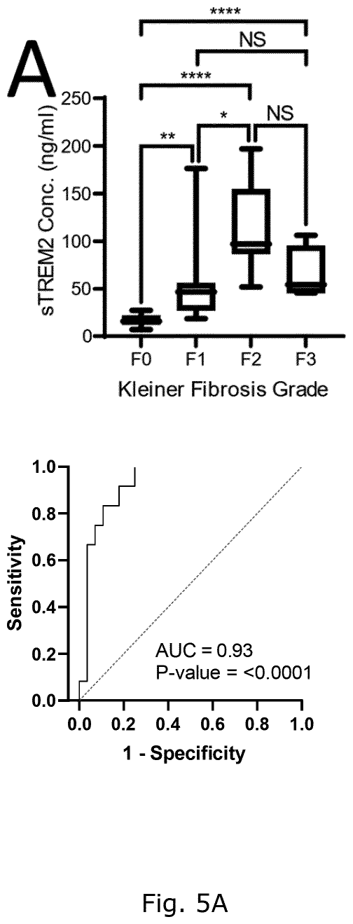

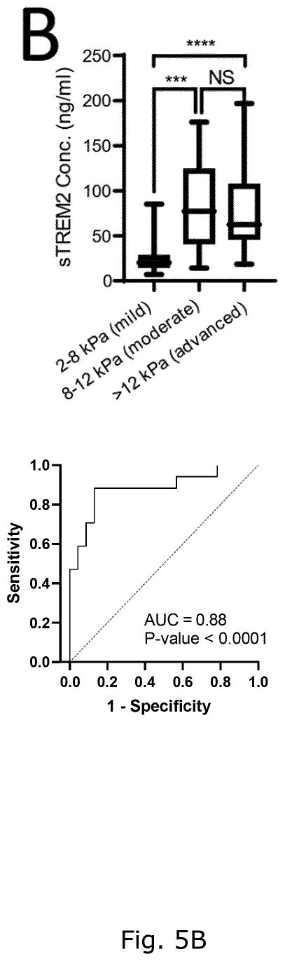

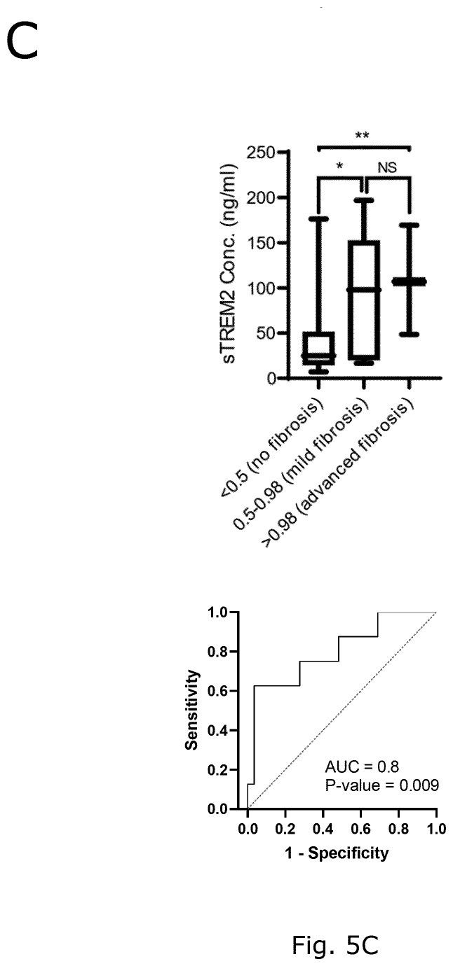

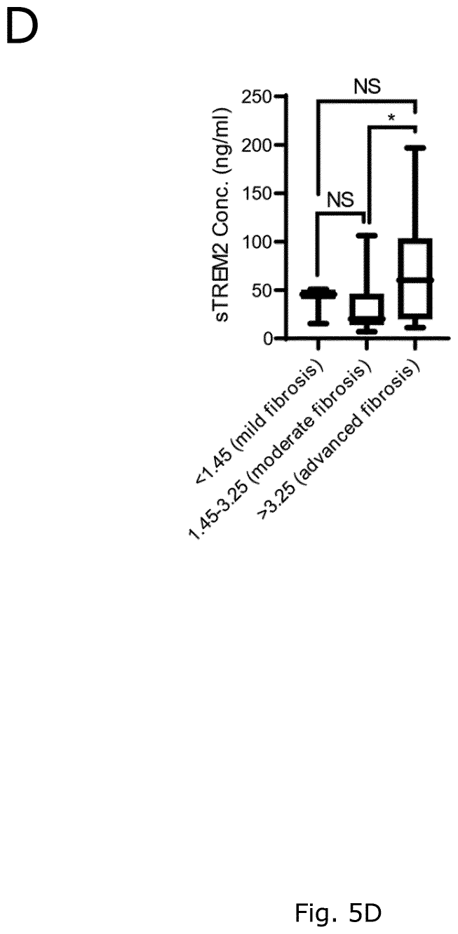

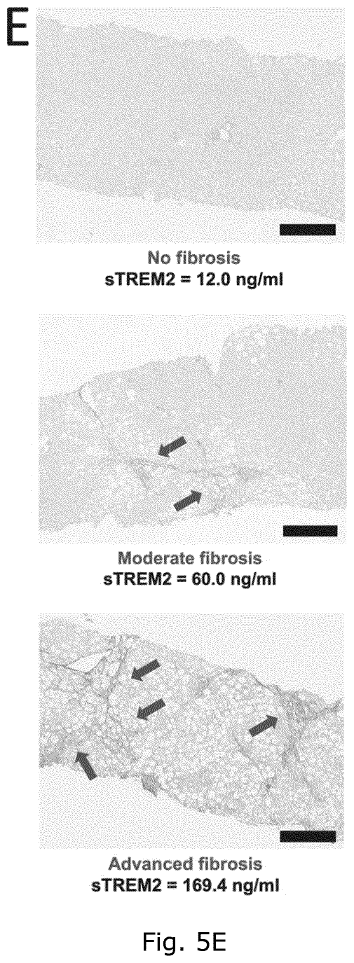

sTREM2 levels were significantly high in F1-F3 patient groups as compared with the F0 no fibrosis group (F0&F1, P=0.001; F0&F2, P<0.0001; F0&F3, P<0.0001) (FIG. 5A). We also noticed significantly elevated sTREM2 levels in periportal as compared with portal fibrosis patient group (F1&F2, P=0.01). However, sTREM2 levels did not differ between bridging fibrosis and portal (F1&F3, P=0.66) or periportal (F2&F3, P=0.09) fibrosis patient groups (FIG. 5A). Then we combined the fibrosis grades (F0-F3) into two groups, F0&F1 (referred to as minimal fibrosis), F2&F3 (referred to as advanced fibrosis) to improve the diagnostic value of fibrosis groups upon comparison. Minimal (n=28) and advanced (n=12) fibrosis patients were strongly discriminated by sTREM2 concentration with an AUROC of 0.93 (95% CI: 0.85-1.0, P<0.0001) and a sensitivity of 92% (95% CI: 0.65-0.99) and specificity of 82% (95% CI: 0.64-0.92) (FIG. 5A). In support of the strong association of sTREM2 levels with Kleiner fibrosis scores, analysis of NASH and no-NASH patient liver tissues revealed strong correlation of fibrosis-related histopathological features with sTREM2 concentrations (FIG. 5E).

Further, categorization of patients according to the FibroScan scores as no-mild fibrosis (2-8 kPa), moderate fibrosis (8-12 kPa), and advanced fibrosis (>12 kPa) groups showed clear discrimination of patients with moderate and advanced fibrosis from no to mild fibrosis group by sTREM2 ( P=0.0002 and P<0.0001 respectively). However, no significant difference was observed in sTREM2 levels between moderate and advanced fibrosis patient groups as identified using FibroScan (P=0.95) (FIG. 5B). Diagnostic accuracy measurement revealed strong discrimination by sTREM2 levels between no-mild and advanced fibrosis groups (moderate and advanced fibrosis combined) with an AUROC of 0.88 (95% CI: 0.77-0.99, P<0.0001) and a sensitivity of 88% (95% CI: 0.66-0.98) and specificity of 87% (95% CI: 0.68-0.95) (FIG. 5B). Additionally, classification of patients with Aspartate transaminase to Platelet Ratio Index (APRI) as no-fibrosis (<0.5), mild fibrosis (0.5-0.98), and advanced fibrosis (>0.98) groups significantly discriminated no fibrosis from mild (P=0.02) and advanced fibrosis patient groups (P=0.006) by their plasma concentration of sTREM2. However, no significant difference was observed between mild and advanced fibrosis patient groups (P=0.71) (FIG. 5C). sTREM2 levels strongly differentiated between the fibrosis (mild and advanced fibrosis combined) and no fibrosis patient groups with an AUROC of 0.8 (95% CI: 0.62-0.99, P=0.009) and a sensitivity of 63% (95% CI: 0.31-0.86) and specificity of 97% (95% CI: 0.83-0.99) (FIG. 5C). Since previous non-invasive evaluation revealed excellent diagnostic ability of sTREM2 to discriminate advanced fibrosis patients, we applied Fibrosis 4 index (FIB-4) that has exhibited strong diagnostic performance in identifying advanced fibrosis among patients with biopsy-proven NAFLD. Patients were categorized as mild fibrosis (<1.45), moderate fibrosis (1.45-3.25), and advanced fibrosis (>3.25) groups. sTREM2 concentration in patients with mild fibrosis did not differ from moderate (P=0.75) and advanced fibrosis patient groups (P=0.31). In contrast to other fibrosis score system classifications, for FIB-4 categorized patients, sTREM2 levels showed significant discrimination between moderate and advanced fibrosis patients (P=0.01) (FIG. 5D) with an AUROC of 0.71 (95% CI: 0.54-0.89, P=0.026) and a sensitivity of 53% (95% CI: 0.31-0.74) and specificity of 90% (95% CI: 0.69-0.98) (FIG. 5D).

### Conclusion

In conclusion, sTREM2 levels can accurately discriminate advanced (grade 3 or higher, F>3) from minimal fibrosis (F<1) patients.

### Example 6—Confirm That Soluble TREM2 Levels in Serum can Also Discriminate NASH From no-NASH Patients

### Aim of Study

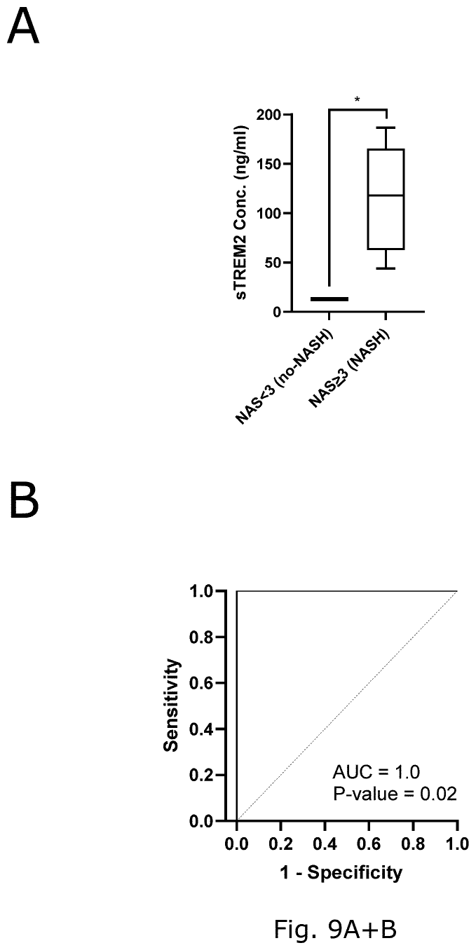

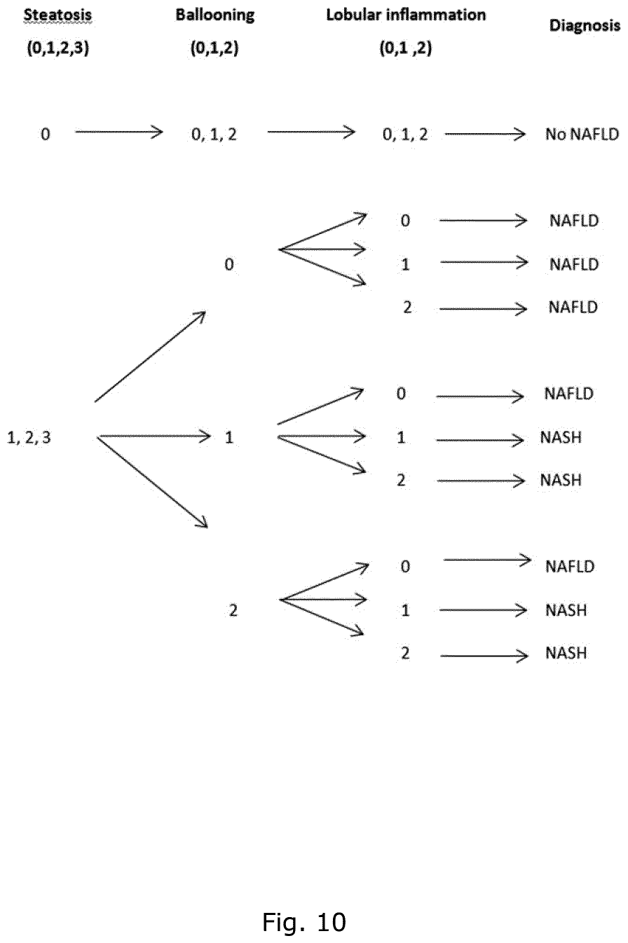

With the aim of exploring the utility of sTREM2 as a non-invasive biomarker to discriminate NASH patients from no-NASH obese controls, we performed quantitative immunoassay (ELISA) on serum extracted from 6 NASH patients and 3 no-NASH obese controls (FIG. 9A).

### Materials and Methods

See example 1.

### Results

sTREM2 was detected in the serum of both the 6 NASH patients and the 3 no-NASH controls above the lower limit of detection (78.1 pg/ml):

| NAFLD activity score | Soluble TREM2 (ng/ml) |
|---|---|
| 8 | 137.85 |
| 7 | 186.70 |
| 6 | 158.50 |
| 5 | 68.71 |
| 4 | 97.10 |
| 4 | 43.89 |
| 0 | 11.90 |
| 0 | 13.26 |
| 0 | 12.76 |

sTREM2 was increased by 9-fold (P<0.02) in NASH patients as compared with no-NASH control groups (FIG. 9A). When patients were dichotomized based on NAFLD activity scores into two groups (cutoff at <3 (no-NASH) and ≥3 (NASH) (16), serum sTREM2 diagnosed NASH from no-NASH patients (P<0.02) (FIG. 9B) with an excellent discriminatory accuracy of AUROC 1.0 (95% CI: 1.0-1.0, P=0.02)

### Conclusion

In conclusion, circulating sTREM2 levels measured in serum effectively discriminate NASH patients (NAS≥3) from no-NASH patients with excellent diagnostic accuracy.

### Example 7—Summary of Data

In this exploratory biomarker study, we selected a well-defined patient cohort from an on-going biopsy-controlled prospective study in obese individuals with BMI>35 and performed quantitative immunoassay to examine TREM2 as a potential soluble biomarker for the non-invasive identification of NASH patients. We found sTREM2 to be an excellent diagnostic discriminator of NASH patients as diagnosed by the histological NAS score. The sTREM2 level was strongly associated with individual histologic features in NASH, i.e., steatosis, ballooning, and lobular inflammation and accordingly, sTREM2 differentiated NASH from no-NASH patients with an AUROC of 1.0 giving an unsurpassed diagnostic value compared to other individual markers. Importantly, we also find sTREM2 to clearly differentiate advanced from no or minimal fibrosis patients (AUROC 0.93) as identified histologically by Kleiner Fibrosis score or validated non-invasive diagnostic indices (liver elastiometry, APRI, and FIB-4).

Liver biopsy is the gold standard for assessment of NASH staging and diagnosis, however the cost and inherent invasive nature of sampling is a major patient compliance challenge (11). To overcome this, a multitude of rapid non-invasive blood-based biochemical markers have been proposed and are at various stages of validation for NASH diagnosis (3). For example, soluble CD163 (sCD163) is a well-known marker for macrophage activation and is associated with tissue inflammation. Considerable interest was generated by studies reporting serum sCD163 to correlate with overall NAS score, steatohepatitis histology, and fibrosis stage in patients with NAFLD albeit with low diagnostic accuracy (12, 13). Similarly, cytokeratin 18 (CK18) which is the most extensively used independently validated test for NASH also has overall modest accuracy with a sensitivity of 66% and specificity of 82% (14). Contrastingly, in our human discovery cohort, sTREM2 individually differentiated NASH patients with an AUROC of 1.0 (FIG. 3D) giving an unparalleled diagnostic accuracy compared to other individual markers including TIMP1 that was previously shown to have the highest diagnostic accuracy to discriminate NASH patients with an AUROC of 0.97 (15) and 0.92 in our analysis, TIMP2 (AUROC 0.51) and MMP2 (AUROC 0.54) (FIG. 7A-C). Meanwhile, a variety of composite biomarkers have also been proposed to increase the diagnostic utility of modest individual markers. These include NASHTest (consisting of a combination of age, gender, height, weight and serum levels of triglyceride, cholesterol, α2-macroglobulin, apolipoprotein AI, haptoglobin, γ-glutamyltransferase, ALT, AST and total bilirubin), AUROC 0.69-0.79 (16); combination of cleaved and intact CK18, adiponectin and resistin, AUROC 0.73-0.91 (17); NASH Diagnostic Panel (comprising diabetes mellitus presence, sex, BMI and serum levels of triglyceride, CK18 fragments and total CK18), AUROC 0.81 (17); and plasma levels of cathepsin D and CK18 combined, AUROC 0.8 (18) (all AUROC values are relative to liver biopsy histological scoring). Intriguingly, the diagnostic accuracy of sTREM2 outperformed all the above-mentioned biomarker combinations for NASH.

Likewise, evaluation of non-invasive biomarkers for fibrosis staging is imperative not only due to the major role as driver for NASH-related clinical outcomes, but also owing to the increased dependence on the inconsistent liver biopsy scoring (3). Introduction of non-invasive scoring system including APRI (AUROC 0.74), FIB-4 (AUROC 0.80) and AST/ALT ratio (AUROC 0.66-0.74) have only managed to moderately rule-in advanced fibrosis (≥F3) patients. In contrast, sTREM2 discriminated advanced from minimal fibrosis patients with an excellent diagnostic accuracy, AUROC of 0.93(FIG. 5A) indicating its potential ability to accurately predict fibrosis-driven NASH clinical outcomes.

### Example 8-—Validation Study in Larger Cohort, Establishing Cut-Offs and Comparison to Other Non-Invasive Biomarkers

### Aim of Study

The value of soluble TREM2 as biomarker of NASH was examined in a larger cohort of obese persons, in which NASH had been graded from liver biopsies. Further, use of levels of TREM2 in blood as biomarker of NASH was compared to other plasma biomarkers of NASH. Finally, an analysis of the data was performed to establish tentative rule-in/rule-out criteria for diagnosis of NASH based on TREM2 levels in blood.

### Materials and Methods

### Study Design and Participants:

Plasma from obese subjects with a moderate to high risk of NAFLD and NASH was analysed. The participants were included via three clinical studies, independently approved by the regional committees on health research ethics and registered at ClinicalTrials.gov (see details, Table 1 below). Participants were recruited from the University Hospital of South Denmark, Esbjerg and Odense University Hospital, Odense. Study 1, patients with severe obesity; Study 2, participants with type 2 diabetes; and Study 3, patients screened for liver disease. Inclusion and exclusion criteria for each study is given in Table 1. All studies were conducted in accordance with the guidelines of the Declaration of Helsinki and the principles of good clinical practice. All subjects gave written, informed consent for study participation and a separate biobank consent.

Validation Cohort (n=170):

We only included patients with an liver stiffness measurement (LSM) ≥8.0 kPa. Patients were derived from three different studies, detail in (Table 1 below): Study 1 (described above, n=59); and Study 2, a liver biopsy-controlled single-center randomized controlled trial in T2DM patients (n=36); and Study 3, a prospective single-center study that has screened subjects for liver disease (Transient elastography (TE) scan and blood samples), and participants with an LSM≥8.0 kPa were offered a liver biopsy (n=75).

### Investigations:

All samples and data were cross-sectionally collected after a minimum of 10 hours of fasting. TE scans were performed by experienced staff using FibroScan 502 touch (Echosens, Paris, France) to obtain a liver stiffness measure (LSM). LSM was performed on the same day as the liver biopsy, except for Study 3, where biopsy date could vary (median 16 days, IQR 28). The lean healthy controls did not receive a liver biopsy, but all had a liver LSM<5.5 kPa (Table 1). Blood was collected by a trained staff, immediately processed and stored at −80° C. for biobanking. All routine biochemical analyses used for calculation of composite biomarkers were performed by a local and central laboratory with commercially available kits.

### Histopathological Assessment

Liver biopsies were performed percutaneously using the Menghini method with a 16-18 G suction needle. Histologic assessments were performed according to NASH Clinical Research Network classification system: steatosis (0-3), lobular inflammation (0-3) and ballooning (0-2). The NAS 0-8 is the sum score of these three features. The steatosis, activity, and fibrosis scoring system (SAF score) was also applied. Hepatic fibrosis was semi-quantitatively assessed according to Kleiner fibrosis stages (1), no fibrosis (F0), perisinusoidal or periportal (F1), perisinusoidal fibrosis in combination with portal and/or periportal fibrosis (F2), bridging fibrosis (F3) and cirrhosis (F4). We defined NASH patients as liver biopsies with NAS≥4.

### ELISA:

TREM2 levels in plasma samples were analyzed using a Human TREM2 ELISA Kit (ab224881, Abcam) according to manufacturer's instructions. Briefly, 50 μL of each standard and sample were added to appropriate wells followed by the addition of 50 μL of antibody cocktail (detector and capture antibody mixture) and incubated for 1 hr at room temperature (RT) with gentle shaking (400 rpm). After washing, 100 μL of 3,3′,5,5′-Tetramethylbenzidine Development Solution was added to all wells and incubated for 10 mins in dark at RT with gentle shaking (400 rpm). Approximately 100 μL of Stop Solution was added to all wells followed by measurement of absorbance at 450 nm in a UV/Vis microplate spectrophotometer (Thermo Scientific, Multiskan GO).

### Statistical Analyses:

All continuous data are presented as medians (±IQR) and categorical variables were described by absolute frequencies and percentages. The significance between two groups was calculated using unpaired two-tailed Student's t-test (parametric data) or Mann-Whitney U-test (nonparametric data). Correlation between two groups were computed with Pearson correlation coefficient (two-tailed p-value. The diagnostic accuracy of plasma TREM2 and other comparative non-invasive tests in the derivation and validation cohort was evaluated using AUROC curves. We determined optimal singular cut-off values for plasma TREM2 by maximizing the Youden index and determined rule-in and rule-out cut-offs by optimizing to 90% specificity and 90% sensitivity, respectively. To test for robustness and relative performance, we did sub-population analyses on each independent cohort. We used a multivariable, ordered logistic regression to evaluate the correlation between plasma TREM2 concentration and NAS as a semi-quantitative variable, to go beyond the dichotomization of NAS done in diagnostic testing. Before the ordered logistical regression analysis, we verified the proportional odds assumption by testing whether the coefficients were equal across categories using a maximum likelihood-ratio test. A p-value of 0.05 was considered statistically significant. STATA 17.0 and GraphPad Prism version 9.1.0.221 were used for statistical analysis and generation of figures.

TABLE 1
Details of participants and methods used in this study.

| | Study 1: | Study 2: | Study 3: |
|---|---|---|---|
| | SEVERELY OBESE | TYPE 2 DIABETES | PATIENTS SCREENED |
| | PATIENTS | (T2DM) PATIENTS | FOR LIVER DISEASE |
| Validation | n = 59 | n = 36 | n = 75 |
| Inclusion | Age 18-70 years | Age 18-79 years | Age 30-75 years |
| criteria | BMI ≥ 35 kg/m² | Diagnosed with | |
| | | T2DM | |
| Exclusion | 1). Chronic liver disease other than NAFLD*. | | |
| criteria | 2). Decompensated cirrhosis^{¥}. | | |
| | 3). Pregnancy. | | |
| | 4). Severe/deadly illness. | | |
| | 5). Hepatotoxic medication^{§}. | | |
| | Only Study 1 and 2\hat{ }: | | |
| | 6). Alcohol use >12 g for women and >24 g for men per day | | |
| | Only Study 2: | | |
| | 7). Following low carbohydrate diet. | | |
| | 8). >10 kg weight loss within the last 3 months. | | |
| | 9). Use of antibiotics within the last 2 months. | | |
| Inclusion | August 2018 | November 2016 | October 2017 |
| start | | | |
| Study name | PROMETHEUS | REDUCTION | SIPHON |
| & ethics | S-20160006G | S-20150217 | S-20170087 |
| approval | | | |

Abbreviations:
US = ultrasound;
TE = transient elastography;
BMI = Body Mass Index
NOTE:
*liver biopsy and biochemical results showing evidence of viral hepatitis, autoimmune liver disease, cholestatic liver disease, primary hemochromatosis;
^{¥}defined as manifestation of ascites, overt hepatic encephalopathy, jaundice, hepatorenal syndrome or variceal hemorrhage;
^{§}tamoxifen, amiodarone, systemic glucocorticoids and methotrexate; [symbol]
Id-number from Ethics committee in Region of Southern of Denmark;
\hat{ }Study 3 included patients with alcohol overuse, however, alcohol overuse patients were excluded in this study.

TABLE 2
Patient characteristics

| | Validation cohort (n = 170) | | | | | |
|---|---|---|---|---|---|---|
| | 1: Severely | | 2: Type 2 | | 3: Patients | |
| | obese | | diabetes | | screened for | |
| | patients | | patients | | liver disease | |
| Study | n = 59 | | n = 36 | | n = 75 | |
| Age (mean, SD) | 45 | (13) | 56 | (10) | 57 | (10) |
| Sex (Male, n %) | 27 | (45.8) | 16 | (44.4) | 46 | (61.3) |

Metabolic phenotype

| | | | | | | |
|---|---|---|---|---|---|---|
| BMI (kg/m²) | 42.7 | (8.6) | 38.0 | (12.1) | 35.0 | (10.3) |
| Obese^{#} | 59 | (100.0) | 29 | (80.6) | 60 | (80.0) |
| Prediabetes^{§} | 25 | (71.4) | 0 | (0.0) | 30 | (85.7) |
| T2DM | 20 | (33.9) | 36 | (100.0) | 32 | (42.7) |
| Dyslipidemia^{γ} | 19 | (32.2) | 17 | (47.2) | 31 | (41.3) |
| Hypertension^{Φ} | 34 | (57.6) | 22 | (61.1) | 44 | (58.7) |

Biochemistry

| | | | | | | |
|---|---|---|---|---|---|---|
| ALT (U/L) | 53.0 | (42.0) | 37.0 | (26.8) | 44.0 | (27.0) |
| AST (U/L) | 36.5 | (28.3) | 27.0 | (16.8) | 33.0 | (24.5) |
| GGT (U/L) | 44.0 | (61.0) | 40.0 | (48.8) | 64.0 | (108.0) |
| Albumin (g/L) | 43.0 | (4.0) | 44.0 | (2.0) | 45.0 | (4.0) |
| Triglycerides | 1.7 | (1.3) | 1.7 | (1.2) | 1.6 | (1.3) |
| Total cholesterol | 4.4 | (1.1) | 4.1 | (0.9) | 4.2 | (1.4) |
| Plasma TREM2 | 55.7 | (62.2) | 47.0 | (39.0) | 41.6 | (40.5) |
| (ng/ml) | | | | | | |
| TREM2 in NAS < 4 | 33.0 | (27.2) | 46.5 | (20.2) | 30.4 | (17.4) |
| TREM2 in NAS ≥ 4 | 86.5 | (81.9) | 74.4 | (51.7) | 63.6 | (39.8) |

Liver assessment

| | | | | | | |
|---|---|---|---|---|---|---|
| TE (kPa) | 11.8 | (10.6) | 11.0 | (3.9) | 11.9 | (8.0) |

| | | | |
|---|---|---|---|
| NAS* group (%) | 14/39/37/10 | 8/56/33/3 | 13/47/31/9 |
| o-1/2-3/4-5/≥6 | | | |
| SAF^{Ø} score (%) | 10/56/34 | 8/67/25 | 12/55/33 |
| no-NAFLD/ | | | |
| NAFLD/NASH | | | |
| Fibrosis stage [symbol] | 9/52/25/9/5 | 3/33/55/6/3 | 3/41/29/20/7 |
| (%) 0/1/2/3/4 | | | |

Abbreviations: BMI, body mass index; T2DM, type 2 diabetes mellitus; ALT, alanine aminotransferase; AST, aspartate aminotransferase; GGT, γ-glutamyl transpeptidase; TE, transient elastography; NAS, NAFLD activity score; SAF, steatosis, activity, and fibrosis.
NOTE:
All descriptive data are medians (±IQR), or counts (%), or else noted in table.
^{#}BMI ≥ 30 kg/m²;
^{§}Fasting glucose between 5.6-7.0 mM, but not T2DM;
^{γ}Taking prescriptions on statins or fibrates;
^{Φ}Taking prescriptions on antihypertensive drugs and stating they have arterial hypertension;
*NASH Clinical Research Network classification system groups;
^{Ø}SAF scoring system; [symbol]
Kleiner fibrosis stages; NA, not applicable.

TABLE 3
Diagnostic accuracy TREM2 in validation cohort and their AUROC,
cut-off, sensitivity, specificity, 95% CI, and p-values.

| | | | | | |
|---|---|---|---|---|---|
| Parameter (NAS ≥ 4 | AUROC | Youden | Sensitivity | Specificity | |
| vs. NAS < 4) | (95% CI) | Cut-off | (95% CI) | (95% CI) | p-value |

Validation cohort

| | | | | | |
|---|---|---|---|---|---|
| Plasma TREM2 - | 0.83 | >47.265 | 0.79 | 0.77 | <0.0001 |
| pooled Validation | (0.77-0.89) | | (0.68-0.87) | (0.68-0.84) | |
| cohort (n = 170) | | | | | |
| Plasma TREM2 - | 0.90 | >56.680 | 0.82 | 0.84 | <0.0001 |
| Study 1, severely | (0.83-0.98) | | (0.64-0.92) | (0.67-0.93) | |
| obese patients | | | | | |
| (n = 59) | | | | | |
| Plasma TREM2 - | 0.84 | >47.265 | 0.73 | 0.87 | <0.0001 |
| Study 3, screened for | (0.75-0.93) | | (0.56-0.86) | (0.74-0.94) | |
| liver disease (n = 75) | | | | | |
| Plasma TREM2 - | 0.65 | >69.545 | 0.54 | 0.87 | 0.14 |
| Study 2, T2DM | (0.45-0.85) | | (0.29-0.77) | (0.68-0.96) | |
| patients (n = 36) | | | | | |
| Plasma TREM2 - | 0.90 | ≥41.000 | 1.0 | 0.71 | <0.0001 |
| Study 1, only T2DM | (0.75-1.0) | | (0.75-1.0) | (0.29-0.96) | |
| subjects (n = 20) | | | | | |
| Plasma TREM2 - | 0.83 | ≥32.000 | 1.0 | 0.64 | <0.0001 |
| Study 3, only T2DM | (0.66-0.99) | | (0.75-1.0) | (0.18-0.90) | |
| subjects (n = 32) | | | | | |

Abbreviations: AUROC, area under the receiver operating characteristic; TREM2, triggering receptor expressed on myeloid cells 2; T2DM, type 2 diabetes mellitus.

TABLE 4
Diagnostic accuracy test probabilities comparison for plasma TREM2,
Fib-4, NAFLD fibrosis score, FAST-score, AST:ALT ratio and Forn's
index with 95% CIs for NAFLD Activity score ≥ 4 in Validation cohort.

| | | | NAFLD | | | |
|---|---|---|---|---|---|---|
| | | | Fibrosis | FAST- | AST:ALT | |
| | Trem2 | Fib-4 | Score | score | ratio | Forns |
| | (n = | (n = | (n = | (n = | (n = | (n = |
| | 170) | 160) | 160) | 159) | 160) | 169) |
| AUROC | 0.83 | 0.58 | 0.54 | 0.78 | 0.56 | 0.55 |
| (95% CI) | (0.77-0.89) | (0.49-0.67) | (0.45-0.76) | (0.71-0.85) | (0.46-0.65) | (0.46-0.64) |
| Rule out | | | | | | |
| Low cut- | ≤38 | ≤1.3 | <−1.455 | ≤0.35 | <0.8 | <4.9 |
| off (ng/ml) | | | | | | |
| n (%) | 58 | 97 | 38 | 31 | 82 | 79 |
| | (34%) | (61%) | (24%) | (19%) | (51%) | (47%) |
| Sensitivity | 90% | 46% | 80% | 94% | 45% | 55% |
| | (81-96) | (34-59) | (68-88) | (86-98) | (33-57) | (43-67) |
| Specificity | 52% | 66% | 26% | 30% | 48% | 48% |
| | (41-62) | (50-76) | (18-37) | (20-40) | (38-59) | (38-58) |
| NPV | 88% | 62% | 63% | 87% | 54% | 60% |
| | (77-95) | (51-72) | (46-78) | (70-96) | (42-65) | (48-70) |
| Grey- | | | | | | |
| zone | | | | | | |
| n | 63 | 58 | 95 | 63 | 61 | 59 |
| | (37%) | (36%) | (59%) | (40%) | (38%) | (35%) |
| Rule-in | | | | | | |
| High cut- | ≥65 | ≥3.25 | ≥0.676 | ≥0.67 | ≥1.2 | ≥6.9 |
| off (ng/ml) | | | | | | |
| n | 49 | 5 | 27 | 65 | 17 | 31 |
| | (29%) | (3%) | (17%) | (41%) | (11%) | (18%) |
| Sensitivity | 54% | 7% | 15% | 66% | 10% | 21% |
| | (41-66) | (2-16) | (7-25) | (53-77) | (4-20) | (12-50) |
| Specificity | 89% | 100% | 81% | 78% | 89% | 84% |
| | (81-94) | (96-100%) | (71-89) | (68-86) | (81-95) | (75-90) |
| PPV | 78% | 100% | 37% | 69% | 41% | 48% |
| | (63-88) | (48-100) | (19-58) | (57-80) | (18-67) | (30-67) |

Abbreviations: PPV, positive predictive value; NPV, negative predictive value.
Determined cut-off for rule-in (diagnosis of at-risk NASH) and rule-out for comparative non-invasive tests have been found in published references. Grey zone refers to intermediate cases not helped in diagnosis by the test.

### Results

### Patient Characteristics:

In total, 4748 participants with risk factors were screened for eligibility to participate in the three studies from which derivation validation cohort of 170 patients with elevated liver stiffness was recruited.

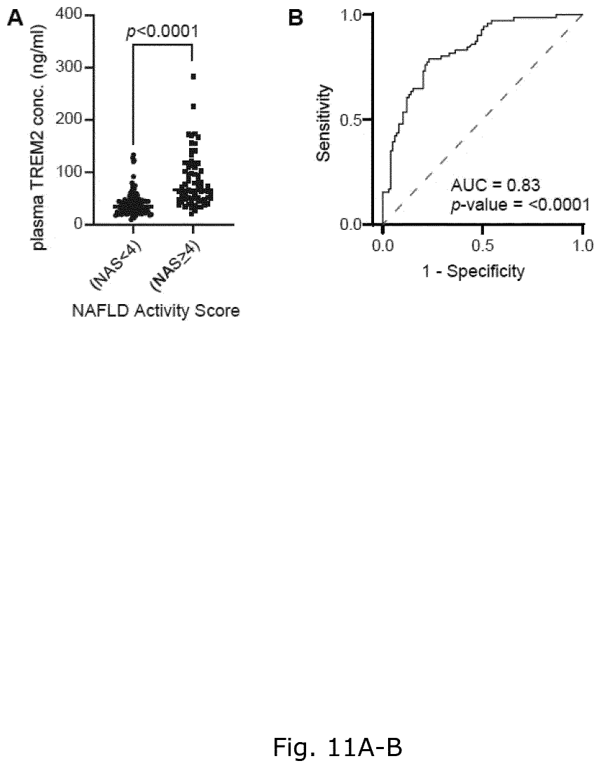

Diagnostic performance of plasma TREM2 in the validation cohort (n=170): Plasma TREM2 was 2.1-fold elevated in NASH patients compared to NAS<4 subjects (FIG. 11A). The plasma TREM2 showed high diagnostic accuracy with an AUROC of 0.83 (95% CI: 0.77-0.89, p<0.0001) to differentiate NASH patients from NAS<4 subjects (FIG. 11B).

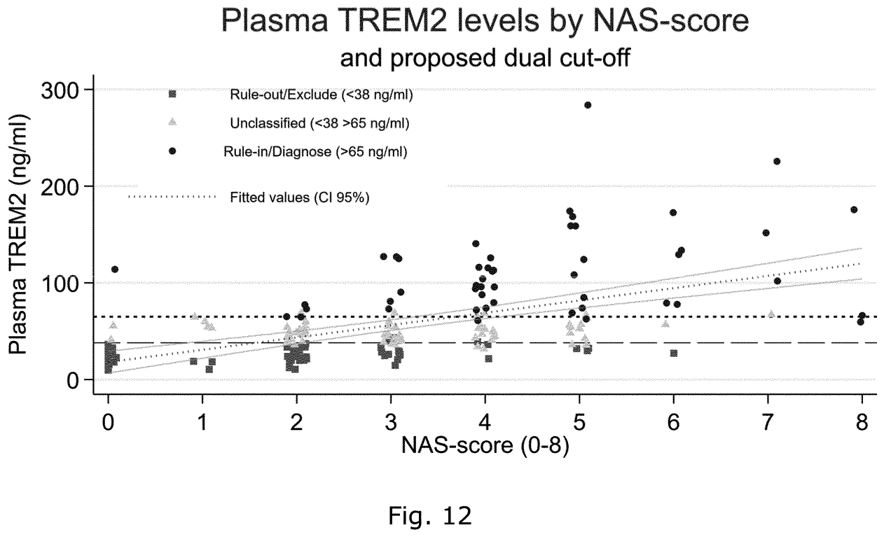

We evaluated a dual cut-off strategy for plasma TREM2 concentration to rule out and rule in NASH. A cut-off at 38 ng/ml was optimal to rule-out (exclude) NASH patients with a sensitivity of 90% (95% CI: 81-96%) and specificity 52%; whereas plasma TREM2 level of 65 ng/ml was the optimal rule-in (diagnose) cut-off with a specificity of 89% (95% CI: 81-94%) and sensitivity 54%. This approach divided the validation cohort in three groups: NAS<4 (n=58), NASH (n=49) and an intermediate group of unclassified patients (n=63, 37%) (FIG. 12).

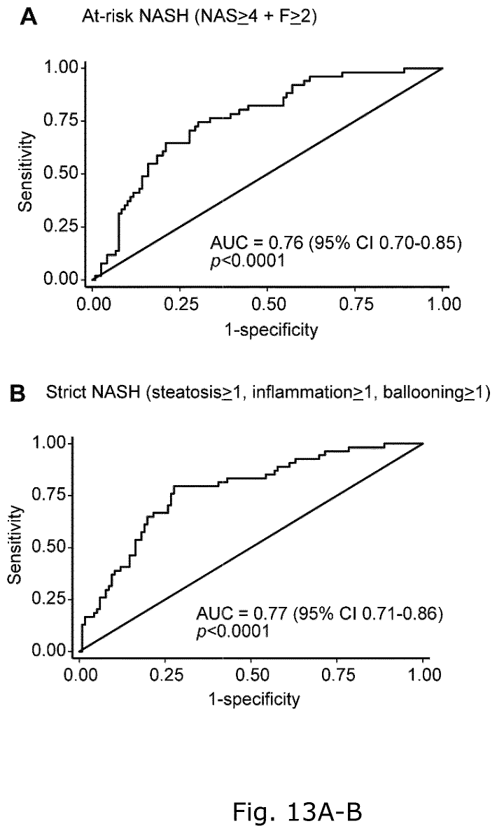

To further validate the ability of plasma TREM2 levels to discriminate patients with NASH, we performed sub-analyses and applied two additional classifications based on different NASH definitions (1) at-risk NASH=NAS≥4 and Fibrosis≥2; and (2) according to SAF-definition, strict NASH =Steatosis≥1, Lobular inflammation≥1, Ballooning≥1. Comparing the three NASH definitions in the 170 participants in the validation cohort, 42% had NAS≥4, 30% had at risk NASH and 32% had strict NASH. The prevalence of NASH according to the different classifications in each cohort can be found in Table 3. Plasma TREM2 levels differentiated at-risk NASH (from no-risk NASH: NAS<4+F≥2, and NAS≥4+F<2, and NAS<4+F<2) and strict NASH (from simple NASH: steatosis<1 (no NAFLD with either inflammation or ballooning or none) and steatosis≥1 (NAFLD with either inflammation or ballooning or none)) with a diagnostic accuracy of 0.76 and 0.77 respectively (FIG. 13).

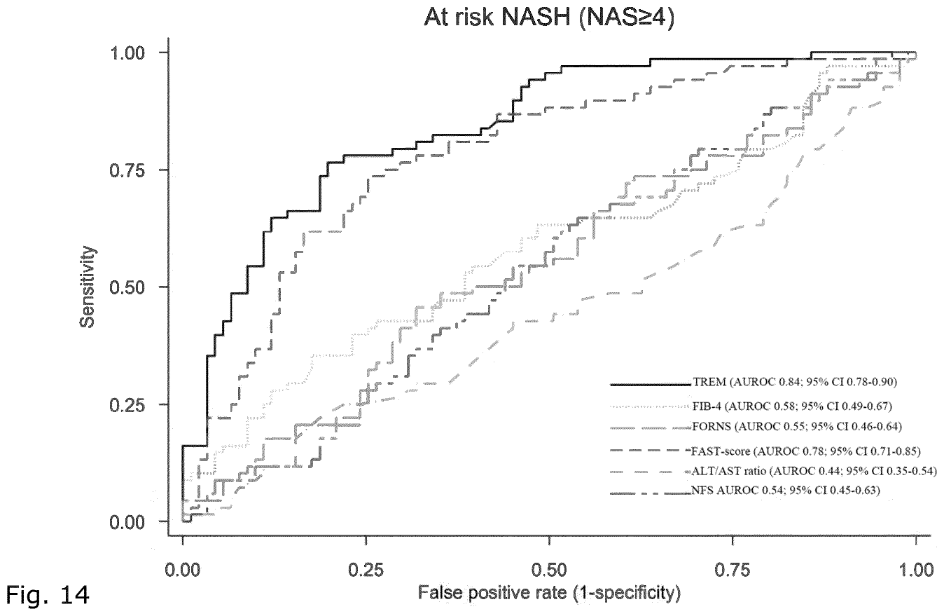

Then, we compared the diagnostic accuracy plasma TREM2 for NASH to other non-invasive test markers involved in NAFLD diagnosis such as Fibrosis-4 (FIB-4), FORNS, AST/ALT, and NAFLD fibrosis score (NFS). In comparative ROC analyses, NAS≥4 plasma TREM2 (AUROC 0.84), performed on par with FAST-score (AUROC 0.78), but out-performed FIB-4 (AUROC 0.58), FORNS (AUROC 0.55), AST/ALT (AUROC 0.56), and NFS (AUROC 0.54) (Table 4 and FIG. 14).

### Association of Plasma TREM2 With NASH Progression and Fibrosis:

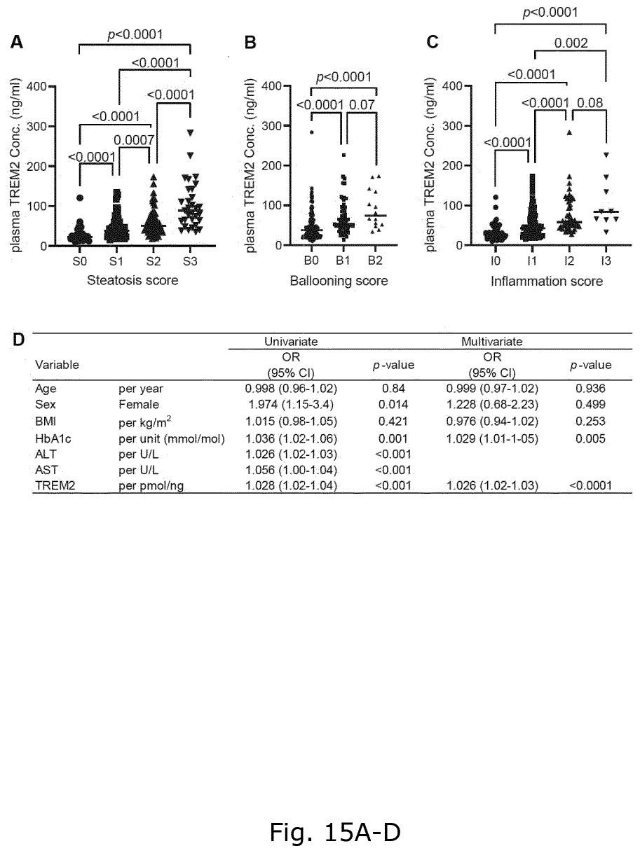

To further understand the association of plasma TREM2 with the different histological components of NASH, we examined the correlation of plasma TREM2 with individual features of steatosis, inflammation, and ballooning. We observed significantly elevated levels of plasma TREM2 in patients with moderate to severe steatosis (grade 1-3), marked ballooning (grade 1 and 2) or moderate to severe inflammation (grade 1-3) as compared to patients with low scores (FIG. 15A-C). Multivariate logistic regression analysis revealed that plasma TREM2 level and HbA1c were independent predictors of NASH in validation cohort (FIG. 15D).

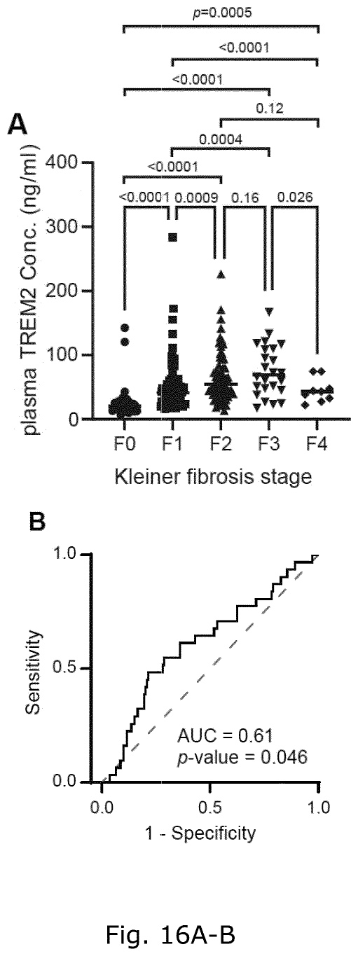

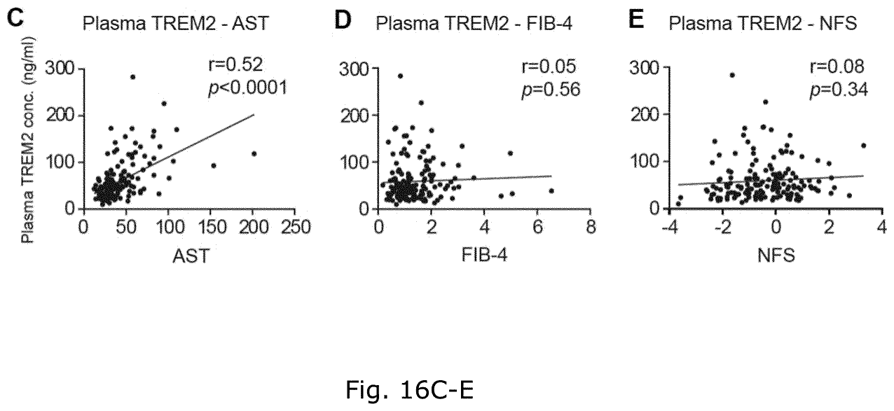

Lastly, we investigated the association between plasma TREM2 and fibrosis. We observed a proportional increase in plasma TREM2 levels according to the fibrosis stages F0-F2, however there was no stepwise increased association of plasma TREM2 levels with fibrosis stages F3&4 (FIG. 16A). The diagnostic accuracy of plasma TREM2 levels to differentiate advanced fibrosis (F3&4) as compared with F0-2 fibrosis patients was low (AUROC 0.61) (FIG. 16B). Then, we tested the association of plasma TREM2 with well-recognized fibrosis markers including AST, NFS, and FIB-4. The plasma TREM2 level exhibited a moderate-to-weak correlation (Pearson) with the plasma AST level (r=0.52), FIB-4 (r=0.05), and NFS (r=0.08) (FIG. 16C-E).

### Discussion of Data

This study evaluated the diagnostic potential of plasma TREM2 as a non-invasive biomarker of NASH in three well-characterized, biopsy-controlled, clinical cohorts, totalling 170 patients with elevated liver stiffness. Although previous studies have found a positive association of Trem2 expression with NASH in liver tissues from patients with NAFLD (steatosis), and the identification of elevated TREM2 levels in plasma and its diagnostic potential to identify NASH has not previously been evaluated. The extrapolation to increased plasma levels of shedded TREM2 is by no-means straight forward due to the complex and unknown mechanism of generation and degradation of soluble TREM2. Surprisingly, we found that plasma TREM2 identified NASH with high diagnostic accuracy (AUROC 0.92). Plasma TREM2 also performed better than other explorative individual biomarkers of NASH such as TIMP1, TIMP2 and MMP2. The plasma TREM2 level was strongly associated with the NAS and individual histologic features (steatosis, ballooning, and lobular inflammation). Further, the proposed rule-in and rule-out cut-off for NASH allowed us to exclude NASH in 34%, and diagnose NASH in 29% of subjects, leaving only 37% of patients for a biopsy.

The ability of plasma TREM2 to identify NASH patients, and its strong association with NAFLD severity makes it an individual biomarker for NASH. When we investigated TREM2 further in NASH progression, we found a strong positive association of plasma TREM2 with hepatocyte ballooning, and inflammation.

### Conclusion

In conclusion, we identify and validate plasma TREM2 as a reliable non-invasive biomarker to diagnose NASH with elevated liver stiffness in a large cohort of patients with histologically characterized NAFLD.

### REFERENCES

1. Anstee Q M, Reeves H L, Kotsiliti E, Govaere O, Heikenwalder M. From NASH to HCC: current concepts and future challenges. Nat Rev Gastroenterol & Hepatol. 2019; 16 (7):411-28.
2. Rockey D C, Caldwell S H, Goodman Z D, Nelson R C, Smith A D. Liver biopsy. Hepatology. 2009; 49(3):1017-44.
3. Wong V W-S, Adams L A, de Lédinghen V, Wong G L-H, Sookoian S. Noninvasive biomarkers in NAFLD and NASH—current progress and future promise. Nat Rev Gastroenterol & Hepatol. 2018; 15(8):461-78.
4. Xiong X, Kuang H, Ansari S, Liu T, Gong J, Wang S, et al. Landscape of Intercellular Crosstalk in Healthy and NASH Liver Revealed by Single-Cell Secretome Gene Analysis. Mol Cell. 2019; 75(3):644-60.e5.
5. Daemen S, Gainullina A, Kalugotla G, He L, Chan M M, Beals J W, et al. Dynamic Shifts in the Composition of Resident and Recruited Macrophages Influence Tissue Remodeling in NASH. Cell Rep. 2021; 34(2):108626.
6. Seidman J S, Troutman T D, Sakai M, Gola A, Spann N J, Bennett H, et al. Niche-Specific Reprogramming of Epigenetic Landscapes Drives Myeloid Cell Diversity in Nonalcoholic Steatohepatitis. Immunity. 2020; 52(6):1057-74.e7.
7. Ramachandran P, Dobie R, Wilson-Kanamori J R, Dora E F, Henderson B E P, Luu N T, et al. Resolving the fibrotic niche of human liver cirrhosis at single-cell level. Nature. 2019; 575(7783):512-8.
8. Govaere O, Cockell S, Tiniakos D, Queen R, Younes R, Vacca M, et al. Transcriptomic profiling across the nonalcoholic fatty liver disease spectrum reveals gene signatures for steatohepatitis and fibrosis. Sci Transl Med. 2020; 12(572):eaba4448.
9. Kleiner D E, Brunt E M, Van Natta M, Behling C, Contos M J, Cummings O W, et al. Design and validation of a histological scoring system for nonalcoholic fatty liver disease. Hepatology. 2005; 41(6):1313-21.
10. Bedossa P, Poitou C, Veyrie N, Bouillot J L, Basdevant A, Paradis V, et al. Histopathological algorithm and scoring system for evaluation of liver lesions in morbidly obese patients. Hepatology. 2012; 56(5):1751-9.
11. Ratziu V, Charlotte F, Heurtier A, Gombert S, Giral P, Bruckert E, et al. Sampling variability of liver biopsy in nonalcoholic fatty liver disease. Gastroenterology. 2005; 128(7):1898-906.
12. Mueller J L, Feeney E R, Zheng H, Misdraji J, Kruger A J, Alatrakchi N, et al. Circulating Soluble CD163 is Associated with Steatohepatitis and Advanced Fibrosis in Nonalcoholic Fatty Liver Disease. Clin Transl Gastroenterol. 2015; 6(10):e114.
13. Kazankov K, Barrera F, Møller H J, Rosso C, Bugianesi E, David E, et al. The macrophage activation marker sCD163 is associated with morphological disease stages in patients with non-alcoholic fatty liver disease. Liver Int. 2016; 36(10):1549-57.
14. Kwok R, Tse Y K, Wong G L, Ha Y, Lee A U, Ngu M C, et al. Systematic review with meta-analysis: non-invasive assessment of non-alcoholic fatty liver disease—the role of transient elastography and plasma cytokeratin-18 fragments. Aliment Pharmacol Ther. 2014; 39(3):254-69.
15. Abdelaziz R, Elbasel M, Esmat S, Essam K, Abdelaaty S. Tissue Inhibitors of Metalloproteinase-1 and 2 and Obesity Related Non-Alcoholic Fatty Liver Disease: Is There a Relationship. Digestion. 2015; 92(3):130-7.
16. Poynard T, Ratziu V, Charlotte F, Messous D, Munteanu M, Imbert-Bismut F, et al. Diagnostic value of biochemical markers (NashTest) for the prediction of non alcoholo steato hepatitis in patients with non-alcoholic fatty liver disease. BMC Gastroenterol. 2006; 6:34.
17. Younossi Z M, Jarrar M, Nugent C, Randhawa M, Afendy M, Stepanova M, et al. A novel diagnostic biomarker panel for obesity-related nonalcoholic steatohepatitis (NASH). Obes Surg. 2008; 18(11):1430-7.
18. Walenbergh S M, Houben T, Hendrikx T, Jeurissen M L, van Gorp P J, Vreugdenhil A C, et al. Plasma cathepsin D levels: a novel tool to predict pediatric hepatic inflammation. Am J Gastroenterol. 2015; 110(3):462-70.

19. Nascimbeni F, Bedossa P, Fedchuk L, Pais R, Charlotte F, Lebray P, Poynard T, et al. Clinical validation of the FLIP algorithm and the SAF score in patients with non-alcoholic fatty liver disease. J Hepatol 2020; 72:828-838.

1. A method for determining the risk of a subject having NASH, the method comprising
A) determining in a blood sample from a subject, the level of soluble TREM2 (sTREM2);
B) comparing said determined level to a reference level; and
determining that said subject is at risk of having NASH, if said level is above said reference level, or
determining that said subject is not at risk of having NASH if said level is equal to or below said reference level; wherein if said subject is determined to be at risk of having or developing NASH a treatment against NASH is initiated.

2. The method according to claim 1, wherein NASH in NASH NAFLD.

3. The method according to claim 1, wherein if said subject is determined to be at risk of having or developing NASH a treatment against NASH is initiated, wherein said treatment is selected from the group consisting of:
treatment with a compound selected from the group consisting of CENICRIVIROC, tropifexor, RESMETIROM, OCALIVA, obeticolic acid, ELAFIBRANOR, ARAMCHOL, IMM124E, SEMAGLUTIDE, liraglutide, LANIFIBRANOR, SELADELPAR, BELAPECTIN, PXL_065, MSDC_0602, ALDAFERMIN, VK2809, EDP_305, HTD1801, PF_05221304, TIPELUKAST, TROPIFEXOR, DF102, LMB763, NITAZOXANIDE, TESAMORELIN, TERN_101, LAZAROTIDE, BMS986036, SAROGLITAZAR, AKR001, CRV431, GRI_0621, EYP0010, BMS_986171, ISOSABUTATE, PF_06835919, PF_06865571, NALMEFENE, LIK066, BIO89_100, NAMODENOSON, MT_3995, PERMAFIBRATE, PXL770, GEMCABENE, FORALUMAB, SGM_1019, KBP_042, HEPASTEM, CER_209, DUR928, SOTAGLIFLOZIN, ELOBIXIBAT, SAR425899, NGM313, NAMACIZUMAB, TERN_201, LPCN_1144, ND_L02_s0201, RTU_1096, IONIS_DGAT2Rx, BEZAFIBRATE, INT_767, NP160, NEULIV, NP135, BFKB8488A, NC_001, VK0214, HM15211, CM_101, AZD2693, NV556, SP_1373, RLBN1127, RYI_018, NVP022, VPR_423, CB4209-CB4211, GKT_137831 or combinations thereof:
OR
treatment by a surgical procedure selected from the group consisting of Bariatric surgery, Roux-en-Y gastric bypass, Gastric sleeve operation and Adjustable gastric band;
OR
treatment in the form of a change of lifestyle, selected from change of diet, exercise, reduction of alcohol consumption, no smoking and/or reduction of smoking;
OR
treatment selected from the group of pharmaceutical compounds approved for treatment of weight loss or hepatic metabolic optimization: metformin, statins, GLP-1 analogues, semaglutide, liraglutide and anti-fibrotic compounds.

4. The method according to claim 1, wherein the subject has a BMI above 25.

5. The method according to claim 1, wherein the sample is a blood plasma sample.

6. The method according to claim 1, wherein the level of sTREM2 is the concentration of sTREM2.

7. The method according to claim 1, wherein the reference level of sTREM2 is at least 28 ng/ml.

8. The method according to claim 1, wherein said method is performed using a method selected from the group comprising immunohistochemistry, immunocytochemistry, Immunoturbidimetry, FACS, ImageStream, Western Blotting, ELISA, Luminex, Multiplex, Immunoblotting, TRF-assays, immunochromatographic lateral flow assays, Enzyme Multiplied Immunoassay Techniques, RAST test, radioimmunoassays, immunofluorescence and immunological dry stick assays.

9. A method of treating NASH in a subject, comprising:
a) receiving an identification of the subject as having a level of soluble TREM2 in a blood sample above a reference level, wherein a level of soluble TREM2 has been identified by a method including a comparison to a reference level; and
b) providing a treatment against NASH to the subject identified as having a level of soluble TREM2 in a blood sample above a reference level.

10. The method according to claim 9, wherein said treatment is selected from the group consisting of:
treatment with a compound selected from the group consisting of CENICRIVIROC, tropifexor, RESMETIROM, OCALIVA, obeticolic acid, ELAFIBRANOR, ARAMCHOL, IMM124E, SEMAGLUTIDE, liraglutide, LANIFIBRANOR, SELADELPAR, BELAPECTIN, PXL_065, MSDC_0602, ALDAFERMIN, VK2809, EDP_305, HTD1801, PF_05221304, TIPELUKAST, TROPIFEXOR, DF102, LMB763, NITAZOXANIDE, TESAMORELIN, TERN_101, LAZAROTIDE, BMS986036, SAROGLITAZAR, AKR001, CRV431, GRI_0621, EYP0010, BMS_986171, ISOSABUTATE, PF_06835919, PF_06865571, NALMEFENE, LIK066, BIO89_100, NAMODENOSON, MT_3995, PERMAFIBRATE, PXL770, GEMCABENE, FORALUMAB, SGM_1019, KBP_042, HEPASTEM, CER_209, DUR928, SOTAGLIFLOZIN, ELOBIXIBAT, SAR425899, NGM313, NAMACIZUMAB, TERN_201, LPCN_1144, ND_L02_s0201, RTU_1096, IONIS_DGAT2Rx, BEZAFIBRATE, INT_767, NP160, NEULIV, NP135, BFKB8488A, NC_001, VK0214, HM15211, CM_101, AZD2693, NV556, SP_1373, RLBN1127, RYI_018, NVP022, VPR_423, CB4209-CB4211, GKT_137831 or combinations thereof:
OR
treatment by a surgical procedure selected from the group consisting of Bariatric surgery, Roux-en-Y gastric bypass, Gastric sleeve operation and Adjustable gastric band;
OR
treatment in the form of a change of lifestyle, selected from change of diet, exercise, reduction of alcohol consumption, no smoking and/or reduction of smoking;
OR
treatment selected from the group of pharmaceutical compounds approved for treatment of weight loss or hepatic metabolic optimization: metformin, statins, GLP-1 analogues, semaglutide, liraglutide and anti-fibrotic compounds.

11. A method for determining the effect of a treatment protocol against NASH for a subject, the method comprising
determining a first level of sTREM2 in a first blood sample from the subject;
determining a second level of sTREM2 in a second blood sample from the subject, wherein the second sample has been obtained at a later time point than the first sample;
wherein the treatment protocol has been initiated or completed before the sampling of the first sample or initiated, continued or completed between the sampling of the first and second sample,
comparing sTREM2 levels in the first sample and the second sample; wherein
a sTREM2 level in the second sample below or equal to the sTREM2 level in the first sample is indicative of the treatment protocol being efficient against NASH; or
a sTREM2 level in the second sample above the sTREM2 level in the first sample is indicative of the treatment protocol not being efficient against NASH.

12. The method according to claim 11, wherein said treatment is selected from the group consisting of:
treatment with a compound selected from the group consisting of CENICRIVIROC, tropifexor, RESMETIROM, OCALIVA, obeticolic acid, ELAFIBRANOR, ARAMCHOL, IMM124E, SEMAGLUTIDE, liraglutide, LANIFIBRANOR, SELADELPAR, BELAPECTIN, PXL_065, MSDC_0602, ALDAFERMIN, VK2809, EDP_305, HTD1801, PF_05221304, TIPELUKAST, TROPIFEXOR, DF102, LMB763, NITAZOXANIDE, TESAMORELIN, TERN_101, LAZAROTIDE, BMS986036, SAROGLITAZAR, AKR001, CRV431, GRI_0621, EYP0010, BMS_986171, ISOSABUTATE, PF_06835919, PF_06865571, NALMEFENE, LIK066, BIO89_100, NAMODENOSON, MT_3995, PERMAFIBRATE, PXL770, GEMCABENE, FORALUMAB, SGM_1019, KBP_042, HEPASTEM, CER_209, DUR928, SOTAGLIFLOZIN, ELOBIXIBAT, SAR425899, NGM313, NAMACIZUMAB, TERN_201, LPCN_1144, ND_L02_s0201, RTU_1096, IONIS_DGAT2Rx, BEZAFIBRATE, INT_767, NP160, NEULIV, NP135, BFKB8488A, NC_001, VK0214, HM15211, CM_101, AZD2693, NV556, SP_1373, RLBN1127, RYI_018, NVP022, VPR_423, CB4209-CB4211, GKT_137831, or combinations thereof;
OR
treatment by a surgical procedure selected from the group consisting of Bariatric surgery, Roux-en-Y gastric bypass, Gastric sleeve operation and Adjustable gastric band;
OR
treatment in the form of a change of lifestyle, selected from change of diet, exercise, reduction of alcohol consumption, no smoking and/or reduction of smoking;
OR
treatment selected from the group of pharmaceutical compounds approved for treatment of weight loss or hepatic metabolic optimization: metformin, statins, GLP-1 analogues, semaglutide, liraglutide and anti-fibrotic compounds.

13. The method according to claim 12, wherein NASH is NASH NAFLD.

14. The method according to claim 1, wherein the method is for determining the risk of a subject for having NASH-associated liver inflammation and/or NASH-associated hepatocyte ballooning and/or NASH-associated Fibrosis.

15. The method according to claim 1, wherein NASH in NASH NAFLD, and the method distinguishes the risk of a subject having NASH NAFLD from subjects having no-NASH NAFLD.

16. The method according to claim 1, wherein the method distinguishes the risk of a subject having NASH from subjects having NAFLD.

17. The method according to claim 1, wherein NASH has a NAS≥3.

18. The method according to claim 1, wherein NASH has a NAS≥4.

19. The method according to claim 1, wherein the subject is a human.

20. The method according to claim 9, wherein NASH in NASH NAFLD.
